(19) United States
(12) Patent Application Publication
(10) Pub. No.: US 2012/0143010 A1
(43) Pub. Date: Jun. 7, 2012

(54) COLLAPSIBLE BARRIER FOR ORGAN RETENTION AND METHOD OF USE

(76) Inventors: Anthony P. Deasey, Severna, MD (US); Steven J. Kubisen, Ellicott City, MD (US); David J. Hicks, Rockville, MD (US); Dennis Smith, Ellicott City, MD (US)

(21) Appl. No.: 13/308,127

(22) Filed: Nov. 30, 2011

Related U.S. Application Data
(63) Continuation of application No. 13/166,635, filed on Jun. 22, 2011.
(60) Provisional application No. 61/392,462, filed on Oct. 12, 2010.

(51) Int. Cl. A61B 1/32

(52) U.S. Cl. 600/207; 600/206

(57) ABSTRACT

A device for human organ packing in a laproscopic surgical procedure, including an expandable barrier configured to be laproscopically implanted into a human abdominal cavity such that a side of the expandable barrier interfaces with organs in the abdominal cavity, thereby forming a barrier between the organs and a region of the abdominal cavity on an opposing side of the barrier relative to the organs, wherein at least a portion of the barrier is configured to be controllably transformed from a first state to a second state, wherein when in the second state the barrier is in a more rigid state relative to the first state.

### CROSS-REFERENCE TO RELATED APPLICATIONS

This application is a continuation application of U.S. patent application Ser. No. 13/166,635 filed on Jun. 22, 2011, which claims priority to U.S. Provisional Patent Application No. 61/392,462, filed on Oct. 12, 2010. In addition, the contents of this application incorporate by reference the subject matter of PCT/US2009/002495 filed on Apr. 22, 2009, and U.S. Provisional Patent Application No. 61/125,219 filed on Apr. 23, 2008. The contents of all of these applications being hereby incorporated herein by reference in their entirety.

### BACKGROUND

Abdominal and pelvic laparotomies require displacement and retention of bowels to create adequate exposure to the surgical site, a procedure commonly referred to as bowel packing. When a section of bowel protrudes from the dressings into the surgical site, the entire bowel packing procedure must be completely redone. Continuous slipping of the bowels into the surgical field results in a waste of time and resources for the surgeon and extended anesthesia time for the patient.

The current bowel packing procedure used in the operating room today is time-consuming relative to the overall priorities of events in a surgery. The surgeon manually displaces the bowels away from the surgical site. Intra-abdominal sponges and towels are then used to pack the bowels or other organs in their displaced location. Surgical cotton sponges are produced in a multitude of forms such as those described by U.S. Pat. Nos. 3,971,381; 4,490,146; 4,626,251; 4,205,680; 4,515,594; and 4,704,109. These cotton sponges are highly absorbent and ubiquitous in modem operating rooms. Finally, abdominal retractors are fitted over the dressings with gentle traction to hold them in place.

They typical bowel packing procedure takes up to thirty minutes, and it must be repeated frequently during extended surgical procedures. Also, the sponges and towels can be forgotten in the abdominal cavity. Furthermore, the cotton sponges used to pack the bowels are made of loose cotton fibers that remain in the abdominal cavity even after removal of the sponges. These fibers can promote peritoneal inflammation, a major cause of post-operative adhesion formation. Furthermore, the sponges tend to dry out over the course of the surgical procedure, becoming abrasive and adhesive to the bowels themselves, further contributing to the formation of adhesions, a leading cause of post-operative morbidity.

### SUMMARY

According to one aspect of the present invention, there is a device for human organ packing in a laproscopic surgical procedure, comprising an expandable barrier configured to be laproscopically implanted into a human abdominal cavity such that a side of the expandable barrier interfaces with organs in the abdominal cavity, thereby forming a barrier between the organs and a region of the abdominal cavity on an opposing side of the barrier relative to the organs, wherein at least a portion of the barrier is configured to be controllably transformed from a first state to a second state, wherein when in the second state the barrier is in a more rigid state relative to the first state.

According to another aspect of the present invention, there is a laproscopic surgery procedure including human organ packing, comprising forming a laproscopic incision in the human, inserting a tube through the incision such that a distal end of the tube is disposed in the abdominal cavity of the patient, moving a flexible barrier in a collapsed state out of the distal end of the tube so that the flexible barrier is disposed in the abdominal cavity, remotely flowing fluid to the barrier to transform the barrier from the flexible state to a more rigid state, and positioning the barrier in the abdominal cavity such that the barrier provides a barrier between the organs and a portion of the abdominal cavity on an opposing side of the barrier relative to the organs.

According to another aspect of the present invention, there is a device for human organ packing in a laproscopic surgical procedure, comprising, an expandable barrier configured and dimensioned to, in an expanded state, be located in a human abdominal cavity such that a side of the expandable barrier interfaces with the organs in the abdominal cavity, thereby forming a barrier between the organs and the portions of the abdominal cavity on an opposing side of the barrier relative to the organs, wherein the barrier is configured to receive a fluid such that introduction of a fluid into the barrier when the expandable barrier is in a collapsed state causes at least a portion of the expandable barrier to expand.

### BRIEF DESCRIPTION OF THE DRAWINGS

FIGS. 1A-1E depict various views of a barrier according to some embodiments detailed herein;

FIG. 2A depicts a frame of reference of the abdominal cavity of an adult human;

FIG. 2B depicts an isometric view of a barrier according to an embodiment;

FIGS. 3A and 3B depict, in graphical format, exemplary comparison information on compression and tension for various silicone polymers;

FIG. 4 depicts, in graphical format, exemplary results for tearing information for various silicone polymers;

FIGS. 5A and 5B depict, in graphical form, comparisons of bowel packing time of cotton laparotomy packs and an exemplary embodiment of a battier as applied in pigs with a 18 cm vertical incision or a 12 cm vertical incision;

FIG. 6 depicts, in graphical form, bowel retention time using cotton laparotomy packs and an exemplary barrier as detailed herein;

FIG. 7 depicts, in graphical form, the change in intra-abdominal temperature over time with cotton laparotomy packs and an exemplary barrier as detailed herein;

FIG. 8 depicts a pack according to another exemplary embodiment;

FIGS. 9A and 9B depict exemplary details according to an exemplary pack;

FIGS. 10 to 13 depict exemplary details according to an exemplary pack;

FIG. 14 depicts intermeshing of protrusions according to an exemplary pack;

FIG. 15 depicts exemplary details according to an exemplary pack;

FIGS. 16 and 17 depict an exemplary inflatable pack;

FIGS. 18 and 19 depict exemplary valve systems of an exemplary inflatable pack;

FIGS. 20 and 21 depict a pack according to yet another exemplary embodiment;

FIGS. 22A and 22B depict a pack according to yet another exemplary embodiment;

FIGS. 23 and 24 depict a pack according to yet another exemplary embodiment;

FIG. 24A depicts an exemplary fluid channel system for a pack according to an exemplary embodiment;

FIG. 25 depicts an exemplary trocar and use thereof with an exemplary pack according to an exemplary embodiment; and

FIG. 26 depicts an exemplary funnel used with an exemplary trocar according to an exemplary embodiment.

### DETAILED DESCRIPTION

An embodiment includes a barrier (including a collapsible harrier, as will be described in greater detail below) for bowel packing having an essentially elliptically-shape, essentially symmetrical along a minor axis of the ellipse, wherein the ellipse has a minor axis having a length, a major axis having a length and a perimeter, wherein the barrier has a notch located on the minor axis below the major axis; and the barrier is composed of material comprising an elastomeric compound and the barrier is appropriately sized for retaining the bowels/intestines of a mammal.

In various embodiments, the essentially elliptically-shaped barriers include at least four radial notches in the perimeter, wherein (a) one or more radial notches arranged above the major axis, wherein each notch has a length from the center of the notch at the perimeter to a base of the notch, wherein the notch length is about 30% to about 40% of the length of the minor axis and each notch has a width at the perimeter of the ellipse that is about 10% to about 15% of a length of a major axis of the ellipse; (b) a second radial notch located on the minor axis below the major axis wherein the second notch has a length from the perimeter to a top of the notch that is about 30% to about 40% of the length of the minor axis and a width at the perimeter that is about 32% to about 42% of the length of the major axis; (c) two or more radial notches arranged left and right of the minor axis wherein each notch has a length from the center of the notch at the perimeter to a base of the notch is at least about 5-15% of the length of the major axis; wherein the barrier is composed of one or more elastomeric polymers having an elastic modulus of about 0.1 MPa to about 10 MPa and the barrier is appropriately sized for retaining the intestines of a mammal. pm In various embodiments, the ratio of the length of the minor axis of the ellipse to the length of the major axis of the ellipse is about 0.55 to about 0.65.

In certain embodiments, the barriers can include one, two, three, four, five or more radial notches per (a) of arranged above the major axis, and two, four, six, eight, or more radial notches to the left of the minor axis and one radial notch to the right of the minor axis per (c), wherein the radial notches of (c) are located at or above the major axis and the barrier includes a bend near the line between two essentially symmetrically positioned radial notches of (c) near the line parallel with the major axis along a line near the top of the bottom radial notch. This bend in the barrier, as well as the curvature and flexibility of the flaps created by the notches make the barriers essentially concave.

The barriers provided in some embodiments include radial notches of essentially any shape that are independently selected, Notch shapes include, but are not limited to V-shaped, U-shaped, and bell-shaped.

Some embodiments include barriers having varying thicknesses. In an embodiment, the portion of the barrier at an intersection of the major axis and the minor axis is greater than the thickness of the barrier at the perimeter. The greater thickness of the barrier in the central portion for contacting the bowel provides greater rigidity, whereas the thinner, more flexible flaps allow for proper positioning of the barrier within the gut.

In some embodiments, the barrier includes a coating. The coating can be useful to prevent sticking of the barrier to the bowel or to provide any other desirable surface property to the barrier. Many appropriate coatings can be selected by one of skill in the art.

Some embodiments provide a barrier including an essentially rectangular body including a first long edge opposite a second long edge and a first short edge and a second short edge wherein the first and second short edge separate the long edges; wherein a) the first long edge is contiguously joined along the length of the first long edge to a top flap having a height and a width, wherein the top flap includes a cutout from an edge of the flap opposite the long edge of the body to which the flap is attached, wherein the depth of the cutout is at least 80% of the height of the top flap; b) the second long edge is contiguously joined along the length of the second edge to a bifurcated bottom flap wherein each half of the bifurcated bottom flap has a height and a width wherein the height and the width of each half flap is essentially the same as the height and width of the other half flap, and the width of the flap extends about 10 to 20% beyond the length of the long edge of body of the barrier on each side of the long edge, wherein the bottom flap is bifurcated by a cutout on an edge of the flap opposite the long edge of the body to which the flap is attached wherein the width of the cutout is about 30% to about 55% of the length of the long edge of the barrier and the cutout is centered along the long edge of the body of the barrier, and the height of the cutout is about 25% to about 40% of a height of the barrier at the tallest paint perpendicular to the first long edge of the body; and c) a first short edge contiguously joined along the length of the first edge to a first side flap and a second short edge contiguously joined along the length of the second short edge to a second flap wherein the first side flap and the second side flap have a height and a width, and each the height and the width of the first side flap are about the same as the height and the width of the second side flap; wherein the width of each side flap is about 10-20% of an overall width of the barrier, wherein the lower edge of each side flap forms a smooth edge with. a bottom half flap; the barrier is appropriately sized for bowel packing in a mammal; and the barrier is composed of one or more elastomeric polymers having an elastic modulus of about 0.1 MPa to about 10 MPa.

In an embodiment, the ratio of the short edge of the rectangular body to the long edge of the rectangular body of the barrier is about 0.2 to about 0.3.

In an embodiment, the barriers are essentially concave. The concave shape of the barrier can be provided in part by a bend near the line that forms the top of the rectangular body, fir example along a line near the top of the notch of (b).

Some barriers include radial notches of essentially any shape that are independently selected. Notch shapes include, but are not limited to V-shaped, U-shaped, and bell-shaped.

Some barriers according to some embodiments have varying thicknesses. In an embodiment, the portion of the barrier at an intersection of the major axis and the minor axis is greater than the thickness of the barrier at the perimeter. In certain embodiments, the thickness of the bifurcated bottom flap is greater than the thickness of the top flaps. The greater thickness of the barrier in the central portion for contacting the bowel provides greater rigidity, whereas the thinner, more flexible flaps allow for proper positioning of the barrier within the gut.

In an embodiment, the barrier includes a coating. The coating can be useful to prevent sticking of the barrier to the bowel or to provide any other desirable surface property to the barrier. Many appropriate coatings can be selected by one of skill in the art.

Some barriers comprise any material having appropriate physical properties as provided herein. Exemplary materials for use for manufacture of the barrier include, but are not limited to elastomeric compounds including various forms of silicone, liquid silicone rubber (LSR), polydimethylsiloxane (PDMS), styrene butadiene rubber, styrene butadiene styrere (SBS) rubber, nitrile rubber, and polychloroprene. Barriers may also include other materials such as fibers encased within the elastomeric compound or radio-opaque substances. Barriers can be manufactured from composites of elastomeric materials.

Some embodiments include methods of use for the barriers for retaining the bowel outside of the surgical field during abdominal surgery. Methods include contacting a bowel of the subject with the barrier to retain the bowel away from a surgical site in a subject, and maintaining the barrier against the bowel during the abdominal surgery. In embodiments of the barrier including a concave face, the bowel is contacted with the concave face of the barrier,

An embodiment of the present invention provides for increasing intra-abdominal temperature during the abdominal surgery and reducing the time required for bowel packing relative to the time required with the use of laparotomy pads.

As used herein, “appropriately sized for bowel packing in a mammal” is understood as being dimensioned to allow for insertion into the abdominal cavity of a mammal including having an face compatible for contact with the bowel for packing and retention, and flaps of a length and width to allow for the barrier to be retained in the abdominal cavity and prevent protrusion of the bowel into the surgical area. For example, in adult humans, the size of the abdominal cavity is about 3.9 to 5.8 inches in the transverse plane at the height of the base of the ribs and about 7.6 to 11.3 inches in the coronal plane at the height of the base of the ribs. An appropriately sized barrier for bowel packing in a mammal having such dimensions is about 5.2 to about 7.5 inches overall height (from ventral to dorsal sides of the abdominal cavity upon placement) and about 8.7 to about 12.5 inches in overall width (from lateral side to lateral side of the abdominal cavity upon placement). The face for contacting the bowel is rectangular with curved corners about 6.26 to about 8.99 inches wide and about 2.60 to about 3.74 inches high with a semi-circular cut-out centered on a long side of the rectangle having a diameter of about 2.80 to about 4.02 inches to accommodate the spine. Therefore, an appropriately sized barrier is overall somewhat larger than the dimensions of the abdominal cavity of the mammal in the transverse plane in which the barrier is to be used, having a face that is somewhat smaller than the dimensions of the abdominal cavity in the transverse plane. Further, the barrier is sufficiently small to allow for compacting of the barrier for insertion into an incision, and manipulation of the flaps to position the barrier in the mammal snugly securely against the bowel.

In some embodiments, features, barriers, or methods provided herein can be combined with one or more of any of the other features, barriers, and methods provided herein.

In some embodiments, the barriers allow for a reduction in operating room time, decreasing cost; a reduction in anesthesia time, reducing surgical risk to the patient; and a reduction in post-operative morbidity associated with the use of surgical sponges used in current procedures. Specifically, the barriers to encompass bowels without leaving behind particulate matter as occurs with sponges.

Some embodiments provide a barrier that is preferably a continuous, one-piece barrier that permits retention of the internal organs away from the surgical site during open abdominal surgeries to prevent obstruction during open surgeries. The barrier is preferably essentially symmetrical relative to the sagittal plane, as the abdominal cavity is essentially symmetrical relative to the sagittal plane. The barrier is essentially elliptical shape as the barrier is designed to fit snugly in the interior of the abdominal cavity which has an essentially elliptical shape. The top portion of the ellipse for contacting the ventral side of the subject is typically more narrow than the bottom portion of the ellipse for contacting the ventral side of the subject. The ventral side includes a cut-out in the center of the bottom edge to accommodate the spine of the subject.

In an embodiment of the invention, the barrier is manufactured from a clear elastomeric compound, preferably a polymer such as a silicone polymer barrier having an overall essentially elliptical shape, with a cut-out to accommodate the spine. The barrier is preferably essentially concave. The barrier is made of one or more elastomeric compounds that have physical properties (e.g., elastic modulus, flexibility/rigidity, tear resistance, etc) to allow the barrier to be easily compressed for insertion into a subject through an incision, but rigid enough to expand once placed in the abdominal cavity and to retain the bowel in conjunction with one, preferably two, retractors. The measurement of the barrier is about the same as the size of the interior of the abdominal cavity or a bit larger, preferably about 2% larger, about 5% larger, about 7% larger, about 10% larger, about 12% larger, or about 15% larger. The relative size of the barrier in each dimension as compared to the abdominal cavity is determined independently. The barrier includes at least one cut-out, preferably a wide cut-out such a semi-circular; U-shaped, or bell-shaped cut out appropriately sized to accommodate the spine of the subject in which the barrier is to be used, about 30% to about 40% of the overall width of the barrier, and about 30-40% of the overall height of the barrier. When positioned in the abdominal cavity of a subject, the barrier is typically held in place by retractors. A sufficient portion of the edges of the barrier are in contact with the interior of the abdominal cavity to prevent protrusion or leakage of the bowel into the surgical site for at least one hour. Methods to test barriers for to determine if they meet the criteria of the invention are routine and known in the art. Methods are provided in the examples below.

The barrier can include further radial notches along the edge of the barrier creating tabs between the notches to be tucked cephally to facilitate positioning of the barrier in the bowel. The notches can be essentially any shape, e.g., V-shaped, U-shaped, bell-shaped around the periphery of the barrier. The notches are of a sufficient depth such that when all of the flaps created by the notches are folded in, the body of the barrier is slightly smaller than the abdominal cavity in which the barrier is to be used. The length of the notches should be limited so as to retain a face on the barrier for contacting the bowel that is at least about 60% of the height of the abdominal cavity (Ventral to dorsal) and at least about 75% of the width of the abdominal cavity (lateral side to lateral side. Typically, the shorter the notches, and therefore the shorter the flaps created by the notches, the more rigid the face of the barrier should be. The greater the number of notches, the more narrow the notches should be to prevent the formation of gaps that can allow the bowel to leak into the surgical field. Moreover, the number of notches is sufficient to allow the flaps to be positioned inside the subject, but few enough to maintain sufficient rigidity in the flaps to retain the barrier in position in the subject.

In an embodiment, the barrier includes a wide notch in the bottom edge of the barrier, creating a bifurcated flap, to provide space for the spine, and one or more relatively deep notch(es) in the top edge of the barrier, typically arranged symmetrically relative to the medial sagittal plane. The deep notch in the top flap allows the top flaps to be tucked in over the bowel on the ventral side of the subject and to provide a point of compression or expansion at the top of the barrier. The bottom of the top flaps is further defined by relatively shallow and narrow notches in the sides of the barrier. The side notches facilitate bending of the barrier to allow the top flaps to be tucked in and for the edges of the top flaps to be pulled in towards the center of the barrier towards the coronal plane. Also defined by the side notches, and continuous with the bottom flaps, are two side flaps. Additional notches can be present in the side flaps. The side flaps are tucked into the sides of the abdominal cavity towards the coronal plane, and the bottom portion of the flaps is tucked cephally to retain the bowel. Bending and positioning of the flaps is preferably facilitated by manufacturing the barrier such that the central body of the barrier for placement against the bowel is more rigid than the flaps. Increased rigidity is typically accomplished by making the central portion of the barrier thicker than the flaps. However, increased rigidity can also be accomplished by the use of a different elastomeric compound, including a composite compound or material, including incorporation of fibers or other materials into the barrier to increase rigidity. Rigidity can also be increased by the use of a frame partly or completely surrounding the central portion of the barrier. Also, the barrier preferably has an overall curved shape with the concave face of the barrier contacting the bowel and facing cephally surrounded by one or more flaps that can be tucked around the periphery of the abdominal cavity to point cephally.

The barrier includes a main body contoured to fit the interior of abdominal cavity, having an overall essentially elliptical shape with an approximately semi-circular or bell-shaped cut-out creating a bifurcated flap to accommodate the spinal and pelvic structures. The body of the barrier and the peripheral flaps are appropriately sized to allow for packing of the bowel in a range of abdominal cavity shapes and sizes as found in vivo. The overall size of the barrier is slightly larger than the cross-section of the interior of the abdominal cavity where it is to be applied, about 2%, about 5%, about 7%, about 10%, about 12%, or about 15%, such that the barrier will expand and exert slight pressure against the interior of the abdominal muscular wall. The barrier can be held in place by tension; however, use of at least one, preferably two or more retractors to retain the barrier in position is exemplary.

Some embodiments of the barriers may be made in different sizes for use in patients of different sizes (e.g., children and adults). Further, it is understood that the barrier can be made for use in non-human animals, for example in pets and other domesticated and non-domesticated. animals of value to humans, for example cats, dogs, non-human primates, animals used for medical research including surgical research such as pigs, zoo animals, etc. The disclosure provides information regarding the exemplary sizes of the barrier for use in adult humans, and the size of the abdominal cavity of adult humans. Provided with this information, a barrier can be made that is appropriately sized for use in mammals other than human adults. Such modifications are well within the ability of those of skill in the art.

The peripheral flaps, continuous with the main body and projecting from the periphery of the barrier aid in the retention of the bowels by forming a pliable seal between the barrier and a range of differently sized and shaped abdominal walls and spinal columns, with larger flaps capable of being pulled across exposed tissue to increase the area covered by the barrier. The flaps may include slits where necessary, such that when the main body is folded, compressed, or rolled, the flaps may be folded, compressed, or rolled in an orthogonal direction. Slits in the flaps run radially from the main body, such that when the barrier is inserted into the abdominal cavity in the final position, the flaps overlap where necessary to prevent protrusion of bowels into the surgical field. The flexibility of the fit of the barrier in the abdominal cavity by an increased number of slits is balanced by the rigidity provided by having extended flaps. In an embodiment, only the flaps for positioning against the ventral and dorsal sides of the abdominal cavity include slits. In an embodiment, the flaps independently include 0, 1, 2, 3, or 4 slits. The rigidity of the body must be balanced with the length and number of the flaps.

The material for the body of the barrier is of sufficient flexibility to permit folding, compressing, or rolling of the barrier to allow for insertion through a retracted incision as small as 10 cm in diameter, while being of sufficient rigidity to expand after folding, compression, or rolling, and retain the bowels for the duration of a surgical procedure when used in conjunction with retractor blades. The barrier is manufactured from one or more elastomeric compounds, preferably an elastomeric polymer such as silicone or polydimethylsiloxane. The barrier can be composed of more than one compound, including composites of one or more elastomeric compounds or polymers, optionally further in conjunction with fiber reinforcement, wherein the fibers are completely contained within the elastomeric compound to modulate structural properties of the barrier, or to provide other properties to the barrier. In an embodiment, the barrier can further include radio-opaque fibers or other radio-opaque materials to make the barrier x-ray detectable.

One type of material usable for a barrier may be silicone based organic polymer, Polydimethylsiloxane (PDMS), which is nonabrasive, inert, and nontoxic in nature. PDMS is optically clear, generally considered to be inert, non-toxic and non-flammable, and flexible enough to allow conformation of the barrier to varying abdominal cavity sizes, yet robust enough to allow interfacing of retractor blades to retain the barrier in the desired position. PDMS has been assigned CAS number 63148-62-9, and is occasionally called dimethicone. The chemical formula for PDMS is (H₃C)₃SiO[Si(CH₃)₂o]ₙSi(CH₃)₃, where n is the number of repeating monomer [SiO(CH₃)₂] units.

In an exemplary embodiment, the main body of the barrier is composed of an inner core of Sylgard® 184 (Dow Corning) polydimethylsiloxane polymer between 8 and 14 mm in thickness, to provide rigidity to the main body, encased in a layer of Sylgard® 186 to confer improved tear-resistance and durability to the barrier. Flaps are made of a tear resistant silicone polymer, with sufficient flexibility to allow for adjustment of the flaps in the abdominal cavity, while providing sufficient rigidity to retain the harrier in place. Exemplary peripheral flap materials include Sylgard® 186 between 2 and 8 mm in thickness, projecting from the main body at angles of between 20 and 60 degrees, and decreasing in thickness with distance from the main body.

When using more than one elastomer is compound for manufacture of the barrier, the compounds can be used together in any manner. For example, a polymer with the desired rigidity can be coated with a polymer having greater smoothness. The body of the barrier can be composed of one polymer, and the flaps can be composed of one or more other polymers to provide varying amounts of rigidity to the body of the barrier and the flaps.

The barriers can also include other components such as coatings to reduce sticking of the barrier to the bowel by coating with polymers, particularly biocompatible polymers, of with commercially available coatings such as Seprafilm®. The coatings may be drug eluting. The coatings may be applied by bulk application, molecular conjugation with the body material, or through nanostructure formation. Examples of possible coatings include: SEPRAFILM®, INTERCEED®, SIROLIMUS®, PACLITAXEL®, EVEROLMUS®, TRANILAST®, DACRON®, SPRAYGEL®, ADHIBIT®, TEFLON®, PRECLUDE® Gore, SyntheMed REPEL-CV®, DuraGen, ADCON™ P (Gliatech), REPEL™ and RESOLVE™ (Life Medical Sciences), INTERGEL™ (formerly LUBRICOAT®), icodextrin, hyaluronic acid, heparin, dextran, tissue plasminogen activator, corticosteroids, non-steroid inflammatory drugs (NSAIDS) such as ibuprophen, chondroitin sulfate, carboxymethylcellulose, dexamethosane, tissue plasminogen including recombinant tissue plasminogen, oxyphenbutazone, collagen, collagen inhibitors, polylactic acid, polyglycolic acid, alginic acid, polycaprolactone, glycosaminoglycans, polyethylene oxide (PEO), polyethylene oxide-polypropylene oxide copolymer in any monomelic ratio (PEG-PPO-PEG), hydroxy ethyl methyl acrylate (HEMA), poly(N-isopropylacrylamide) (NIPAAm), polytetrafluroethylene (PTFE), polyesters, and silane, or modification by radio frequency gas discharge (RFGD), and radiation grafting, polytetrafluoroethylene, (PTFE), polylactic acid, polyglycolic acid, alginic acid, polycaprolactone, glycosaminoglycans, HEMA, ePTFE, polyesters, carboxymethylcellulose, dexamethasone, tissue plasminogen including recombinant tissue plasminogen, oxyphenbutazone, corticosteriods, icodextrin, hyaluronic acid, hyaluronan, and collagen inhibitors. Additionally, the barrier surface can be modified by silanization, RFGD, or radiation grafting.

Alternatively, barriers can be coated with agents, for example, anti-microbial agents such as anti-viral agents or anti-bacterial agents. The use of such agents may be useful for the protection of the subject as well as the surgical staff and to reduce the possibility of transmission of infection from subjects infected with HIV, hepatitis, especially drug-resistant forms of hepatitis, methicillin resistant staphylococcus aureus (MERSA), etc.

The flexibility of the materials used to make the barrier also allow for a single size barrier to be used in subjects through a range of sizes. However, in an embodiment, the barriers are made in different sizes for use in different subjects. The multiple flapped structure of the invention creates surfaces for retaining the organs within the exemplary location of the abdominal cavity and away from the surgical site. Determination and selection of a barrier of an appropriate size for use with a particular subject is well within the ability of those of skill in the art.

The contour of the barrier conforms to the sides of the abdominal cavity, in an essentially concave shape proximal to the bowel when inserted, to prevent protrusion of the bowels into the surgical field and allows the bowels to be scooped cephally in a single, smooth motion. The top flap (for positioning adjacent to the ventral side of the subject) is preferably designed with a one or more vertical slits to fold over the top of the bowels in the abdominal cavity and prevent the bowels from leaking over the top of the barrier, obscuring the surgical field. Preferably the bottom flaps further include at least one slit or opening, both to provide space for the spine, and to allow for the barrier to conform to the abdominal cavity. The conforming of the barrier to the sides and top of the abdominal cavity by the side and top flaps allows for bowel retention for the duration of the surgical procedure and simplify the packing procedure.

Further, in an embodiment, the barriers are made of a clear material which allows the bowels to be monitored continuously throughout the procedure, an advantage not allowed by the laparotomy packs currently being used. Further, the use of an elastomeric material provides for retention of both moisture and warmth in the abdominal cavity as compared to packing methods using surgical sponges as demonstrated herein.

Insertion of the Barrier into the Abdominal Cavity:

Insertion of the barrier into the patient is preferably done manually by the surgeon. The barrier is first compressed sufficiently to allow for insertion through the retracted incision in the patient. The barrier is preferably oriented prior to insertion so that the top flaps will be pointed towards the ventral side of the patient and the concave proximal face of the barrier will face cephally after decompression. Insertion can be facilitated by placing the patient in the Trendelenburg position, a vertical tilt, typically about 15°, with the feet higher than the head. Once the barrier is inserted, it expands against the abdominal side walls and can be used to scoop the bowels cephally by placing the center of the barrier against the bowel and adjusting the flaps cephally. Once this has been completed, the barrier is secured. in place using retractor blades. The exact position of the barrier will depend on the location of the incision and the choice of the surgeon. The exact method of insertion of the barrier is not a limitation of the invention. It is understood that a barrier can be used for packing other organs clear a surgical field. The specific use of the barrier is not a limitation of the barrier.

Removal of the Barrier from the Abdominal Cavity:

When removing the barrier from the patient, the surgeon supports the barrier with one hand while the retractor blades are removed. The surgeon places the index finger of either hand into the notch between the bottom flaps, pulling the barrier upward while compressing it with the palms. The exact method of removal of the barrier is not a limitation of the invention.

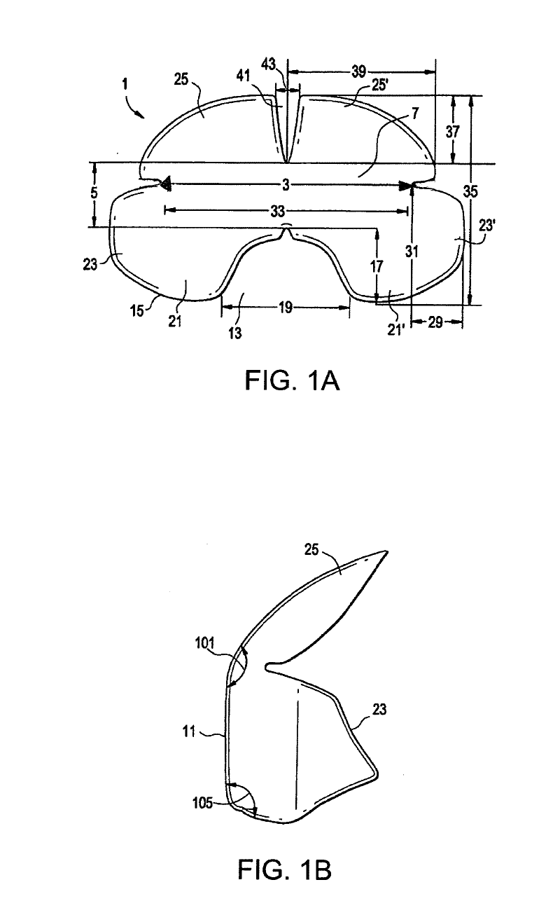

FIGS. 1A to E show an embodiment of the barrier 1 of the invention. Measurements are provided in parenthesis to approximate the exemplary medium size of the embodiment of the invention. The medium size barrier for use in a human adult can vary up to about 5% from the base measurement provided in the detailed description of FIG. 1 below, and the measurement provided should be understood to include this variation within the range of about 0 to about 5% (i.e., about 1%, about 2%, about 3%, about 4%, or about 5%). The barrier for use in a small adult human is within the range of about 75% to 85%, preferably about 80% of the base dimensions provided for the medium size harrier. The barrier for use in a large adult human is within the range of about 110% to 120%, preferably about 115%, of the base dimensions provided for the medium size barrier. One of skill in the art can readily perform the calculations required to determine the appropriate size of the barrier based on the disclosure provided herein.

The specific size of the barrier is not a limitation of the invention. The limitation of the size of the barrier is that it must be appropriately sized for insertion into the abdominal cavity of a subject, preferably a human subject, for bowel retention. The invention provides a barrier, more preferably a single piece barrier, including a body having a two long edges opposite each other on the body, and two short edges between the long edges on opposite sides of the body. The barrier further includes two top flaps on a long edge of the body, two side flaps on short edges of the body adjacent to one long edge, and a cut-out on the long edge opposite the long edge with the flaps. The barrier has a smooth proximal surface for placement against the bowel of the subject to displace the bowel cephally and expose abdominal organs for surgery.

As shown in the front view in FIG. 1A of an embodiment of the barrier 1, the essentially rectangular central portion 207 of the barrier has a width 3 (7.82 inches) and a height 5 (3.63 inches), and in the embodiment shown is the essentially rectangular portion of the barrier defined by a first long edge at the base of the notch separating the first and second top flap of the barrier, and a second long edge defined by the top of the bell-shaped cutout in the bifurcated bottom flap, and short edges perpendicular to the long edges, adjacent to the paired notches on opposing sides of the barrier that separate the top flap from the body of the barrier. The central rectangle 207 of the barrier is not a structural element of the barrier, but instead is used in the description of the barrier herein to allow for the description of the size and position of various components of the barrier.

The body 7 of the barrier serves mainly to support the bulk of the bowel and intestines on the concave proximal face 9, and also interfaces with retractor blades currently used in laparotomies on the convex distal face 11. The standard retractor blade setup uses two blades that interface with the lateral sides of the body. There is a bell-shaped cut 13 with in the bottom edge 15 (for placement towards the ventral wall of the abdominal cavity), opposite the edge including the top flaps. The bell shaped cut has a height 17 (2.28 inches) and a base width 19 (4.00 inches). The cutout is provided to accommodate the ventral medial part of the body in the sagittal plane, and designed to conform to, and provide space for, the spinal cord. The cut also creates two flaps 21 and 21′ on the bottom edge 15 of the barrier. Upon insertion, two side flaps 23 and 23′ contact the lateral sides of the abdominal cavity and two top flaps 25 and 25′ contact the ventral side of the abdominal cavity. These flaps and the body of the barrier are one contiguous structure, preferably made from a single piece of elastomeric material, preferably a silicone polymer. The side flaps curve upward from the proximal face of the body of the barrier (3.71 inches) in the embodiment shown for a desired length 27 and each have a width 29 (1.55 inches) and a height 31 (3.63 inches), in an alternative embodiment, the upward curve of the flaps is shorter, but the material from which the flaps is somewhat more rigid to retain the barrier in the abdominal cavity. The dimensions of the barrier, including the body and the side flaps are width 33 (10.92 inches), height 35 (6.53) and depth 27 (3.71 inches). The side flaps serve to aid the body of the barrier in containing bowels that may protrude around the sides of the barrier in the abdominal cavity otherwise. The top flaps have a height 37 (2.90 inches) and a width 39 (4.75 inches) There is also a U-shaped cut 41 to separate the two flaps 25 and 25′ having a base width 43 (0.86 inches). The top flaps lean towards the proximal face of the body of the barrier at an angle 45 preferably at an angle in a range of about 145° to 155° relative to the body 7 of the barrier. The angle of the flaps to the proximal face becomes more oblique as the length of the flaps becomes shorter to maintain the overall height of the barrier. The purpose of the top flaps 23 and 23′ is to help secure the bowels on the ventral side of the subject. The harder preferably has varying thicknesses throughout the body and flaps. The thickness of the body and flaps is consistent between the varying sizes of the barrier, however, the specific thicknesses of various portions of the barrier depends on the specific material used for the barrier. When the barrier is made from PDMS and the flaps are sized as provided in the detailed embodiment, the top flaps 25 and 25′ are the thinnest, within the range of about 0.14-0.16 inches; the body 7 is the thickest portion, within the range of about 0.60 to about 0.68 inches; and the side flaps 23 and 23′ have an intermediate thickness, within the range of about 0.30 to 0.34 inches. Modifications of thickness of various portions of the barrier is within the ability of those of skill in the art, and depend on a number of factors discussed herein, including, but not limited to the specific material or materials from which the barrier is made.

FIG. 1B shows an embodiment of the barrier as the various portions would be positioned when placed in a supine subject and the subject was viewed from the lateral side. The convex face of the barrier 11 is shown and the concave face of the barrier is hidden by the top 25, side 23, and bottom flaps. The top flap 25 bends cephally at an angle 101 of about 140° to about 160° to the concave face of the barrier. In the embodiment shown, the bottom edge of the bottom flap having a width approximating the height of the bell-shaped cut in the bottom flap folds towards the concave face of the barrier at an angle 105 of about 95° to 115° with the concave face of the barrier with the flap lengths shown. With shorter top and bottom flaps, the angle of the flaps to the proximal face of the barrier becomes more oblique. The side flaps are also positioned cephally towards the concave face of the barrier.

FIG. 1C shows an alternate view of an embodiment the barrier as it would be positioned when placed in a supine subject and the subject was viewed looking down at the subject from above through the ventral side. The top 25, 25′ and side 23, 23′ flaps are angled towards the proximal face 9 of the barrier.

FIG. 1D shows an alternate view of an embodiment of the barrier as it would be positioned when placed in a subject and viewed at an oblique angle looking from the shoulder towards the feet of the subject. The top 25, 25′, side 23, 23′, and bottom 21, 21′ are pointing cephally to define the concave proximal face 9 for contact with the bowel. In the embodiment shown, the relative thicknesses of the various portions of the barrier are shown. The varying thicknesses of the portions of the barrier allow for varying amounts of rigidity or flexibility. The body 7 of the barrier is the thickest portion of the barrier, providing substantial rigidity in the portion of the barrier for contact the bowel. The rigidity of the body may be modified by the inclusion fibers completely enclosed within the elastomeric material of the body, or may be surrounded partially or completely by a frame to modify the rigidity. The decrease in thickness of the barrier in the flaps facilitates manipulation of the flaps. The bottom and side flaps of the barrier are somewhat thicker than the top flaps in the embodiment shown as the patient will likely be in a supine position and the bottom flaps will need to be sufficiently rigid to be retained in position to reduce or eliminate inadvertent contact with the spine. In an embodiment, after proper insertion, the barrier does not contact the spine. The top flaps of the barrier are thinnest and most flexible providing the user with options regarding exact placement of the barrier and providing allowance for variations in size between subjects.

FIG. 1E shows an overlay of an embodiment of the barrier with an ellipse 201, with the major axis 203 and minor axis 205 shown for determining relative sizes of various portions of the barrier. The Figure also shows an overlay of an embodiment of the barrier with a rectangle 207 defining the body of the barrier for determining the relative sizes of various portions of the barrier. The length of the flaps will determine the angle at which the flaps are positioned relative to the proximal thee of the barrier.

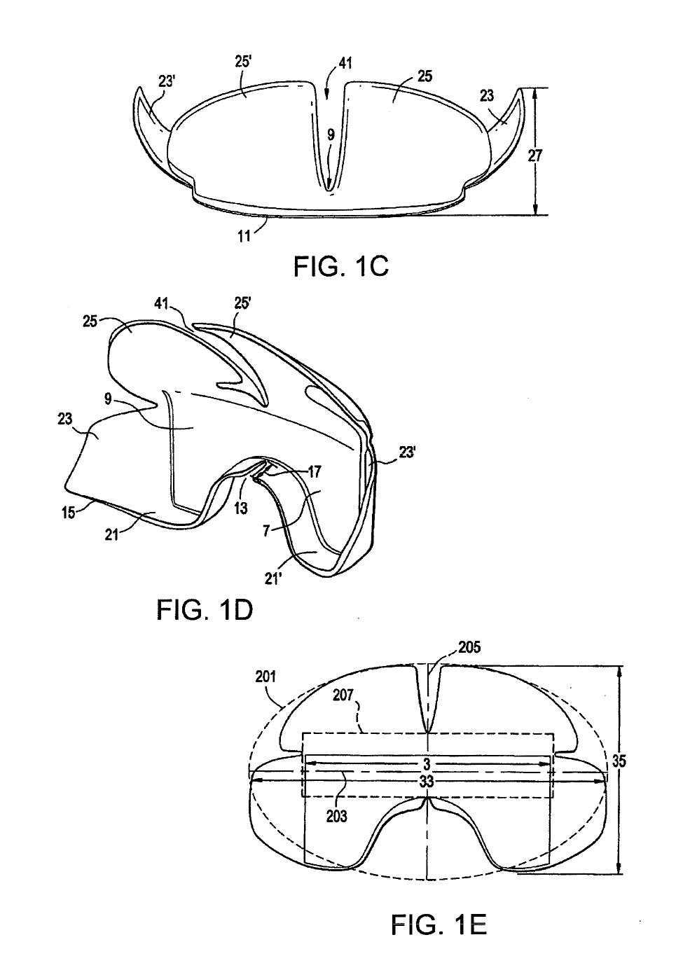

FIG. 2A illustrates dimensions of the average human abdomen that were used to determine the dimensions of the barrier for use in adult human with the transverse 301 and coronal 303 planes indicated. Using the measurements of the adult human abdominal cavity and the barriers, one can easily determine the appropriate dimensions for a barrier for use in a subject other than an adult human provided with the dimensions of the abdominal cavity (human child, dog, cat, other mammal). Anthropologic data was used to determine the small, medium, and large sizes designed to fit at least 95% of the adult human population. This flexibility of the barrier allows it to conform to cavities that may otherwise be too big or too small. In an embodiment, the small size will be about 5.20 inches total in height and about 8.70 inches total in width; the large size will be about 7.50 inches in height and about 12.50 inches in length; the medium size of the barrier is about 6.53 inches in total height, including the body and flaps, and about 10.92 inches wide.

FIG. 2B shows a transparent view of an embodiment of a barrier showing examples of dimensions for the small, medium, and large adult human sizes.

The invention is designed to be interoperable with surgical retractor blades currently used during the bowel packing procedure. For incision sizes between 12 cm and 18 cm, it has been found that the barrier retains the bowels effectively for a 1-hour period without the support of retractor blades.

Additionally, the side and top flaps are designed to conform to the side and top of the abdominal cavity when packing cephally, to the pubic symphysis, so as to expose the lower pelvic cavity. Further the barrier can be modified to enable packing to be performed caudally so as to expose the upper and middle abdominal cavity. Thus, the barrier can be applied to a greater scope of abdominal and pelvic laparotomy procedures that require displacement of the bowels. Modification of the flap length and shape is within the ability of those of skill in the art based on the teachings provided herein.

The invention provides methods and the use of a barrier for achieving certain desirable outcomes during surgery. For example, the use of an elastomeric barrier of the instant invention provides advantages over laparotomy sponges not only in ease of use, but in improved patient outcomes, such as maintenance of intra-abdominal temperature and moisture during surgical procedures as demonstrated in the Examples below. The use of the barriers provides a method for observation of the packed bowel during surgery as the barrier is preferably clear. During surgery, the bowel can be observed through the barrier for discoloration, bleeding, or other undesirable events.

Use of barriers also provide for a reduction in adhesion formation as a result of bowel packing as compared to bowel packing performed with laparotomy sponges. Adhesions are due at least in part to fibers from laparotomy sponges that remain in the abdominal cavity after the removal of sponges at the end of the surgery. As the barrier includes no exposed fibers, none can be left behind, eliminating at least one substantial cause of adhesions. The barrier also reduces the possibility of medical errors as the barrier is a single piece that would be difficult to overlook at the conclusion of a surgical procedure, unlike a laparotomy sponge.

The use of the barriers also provide a method to decrease bowel packing time and in longer surgeries the number of times that the bowel needs to be packed, thereby decreasing surgical time. The overall surgical time reduction will depend on the total surgical time and the number of times the bowel would need to be packed. However, the invention provides methods to reduce bowel packing time by at least 10%, at least 20%, at least 25%, at least 30%, or at least 35% as compared to bowel packing with laparotomy sponges.

### EXAMPLE 1

### Manufacture of a Barrier

The prototype barrier was cast using a combination silicone and Plaster of Paris™ mold. PDMS Sylgard® 186 was used to increase tear resistance of the barrier. The main body of the barrier was reinforced with PDMS Sylgard® 184 to prevent buckling upon application of shear and transverse forces by surgical retractor blades. The current prototype was scaled to fit a medium-sized human adult based on dimensions obtained from anthropologic data of pelvic size distributions.

### EXAMPLE 2

### Material Testing Results

Two exemplary materials were tested to select the better material and the appropriate ratio of cross-linker for casting of a prototype. The barrier can be made of any elastomeric material having the desired elastic modulus, compression, and tear resistance and strength that can be sterilized, and that is preferably clear or at least translucent. Determination of properties of materials for making the barrier in the invention can be determined empirically using routine methods such as those provided below. Alternatively, the properties of the materials are commonly provided in catalogs, specification sheets, technical bulletins, and other manufacturer's information. Exemplary materials for use for the barrier have a Young's modulus between 0.1 MPa and 10 MPa and a yield point of 250 KPa-5 Mpa. Materials should be tear resistant when subjected to 100 N of shear force. A single round of sterilization should not cause the material to lose these properties. The elastomeric polymer for manufacture of the barrier can include more than one polymer, either a mixture of polymers, different polymers for different portions of the barrier, or coating one polymer with another polymer. Such selections are a matter of choice and can be made by one of ordinary skill in the art.

Determination of Elastic Modulus

The elastic moduli of Sylgard® 184 and Sylgard® 186 were tested using routine methods to determine if the materials have elastic moduli in a range that offers sufficient flexibility for ease of insertion of the barrier and sufficient rigidity of the barrier to effectively retain the bowels. The target, range of elastic moduli was between 0.1 MPa and 10 MPa. Base to cure ratios of Sylgard® 186 were varied in order to further explore the possible ranges of elastic modulus values that can be obtained. In addition, stress-strain curves were determined to determine the yield point of each material.

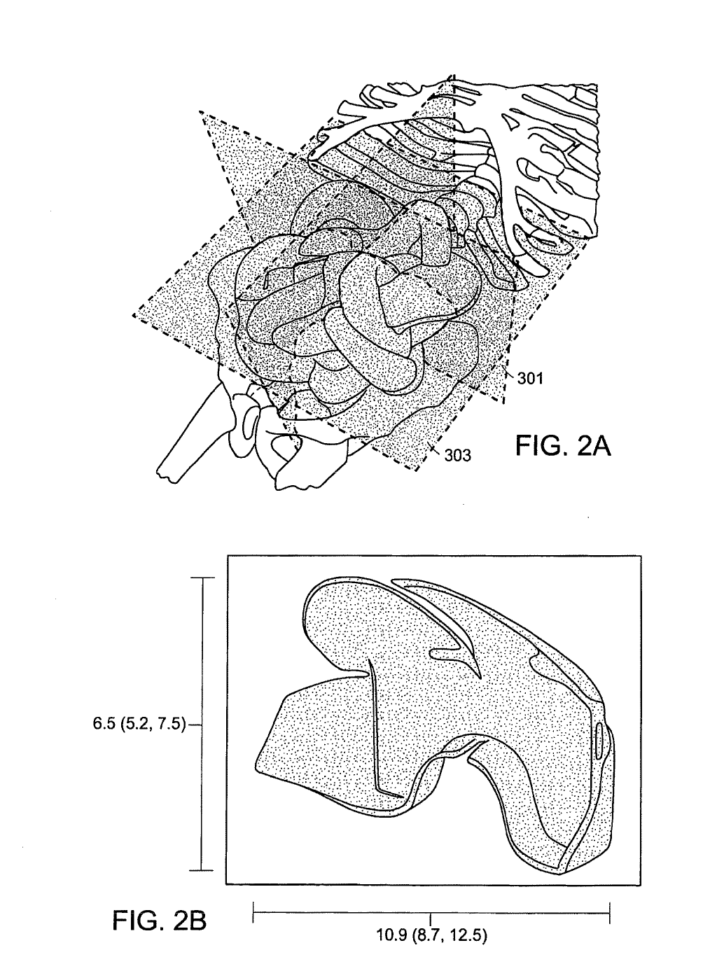

Five samples each measuring 2″×2″ of Sylgard® 184 and 186 were tested and results averaged. The test equipment included an ATS (Applied Test Systems®; Butler, Pa.) Series 1601 and a Computer-Controlled Universal Test Machine (UTM). The results obtained are provided in FIGS. 3A and B.

It was determined that Sylgard® 184 and 186 have elastic moduli of 14 KPa and 2.9 KPa respectively; this falls within the range appropriate for use in barriers. Because of its increased yield point of 900 KPa versus 500 KPa, Sylgard® 186 was identified as a better material choice than Sylgard® 184. Finally, a base to cure ratio of 10:1 was chosen for Sylgard® 186 because this ratio exhibited the highest elastic modulus of the samples tested. However, it was well below the lower limit of 0.1 MPa.

2. Determination of Tear Resistance and Tear Strength

Tear resistance is the resistance of a material to initial tearing while tear strength represents the force required to tear a pre-slit material, An un-slit material needs to have no visible tears develop upon application of 100 N of shear force. The amount of shear force required to tear pre-slit material was also determined to further understand one possible failure mode of the harrier. In order to determine if Sylgard® 184 and or Sylgard® 186 would be able to withstand the shear forces applied by the retractor blades on the body of the barrier, both tear resistance and tear strength of the material were determined. Sylgard® 184 and 186 were compared to determine the most tear-resistant material. Force thresholds were determined from measurements made in a simulated abdominal cavity.

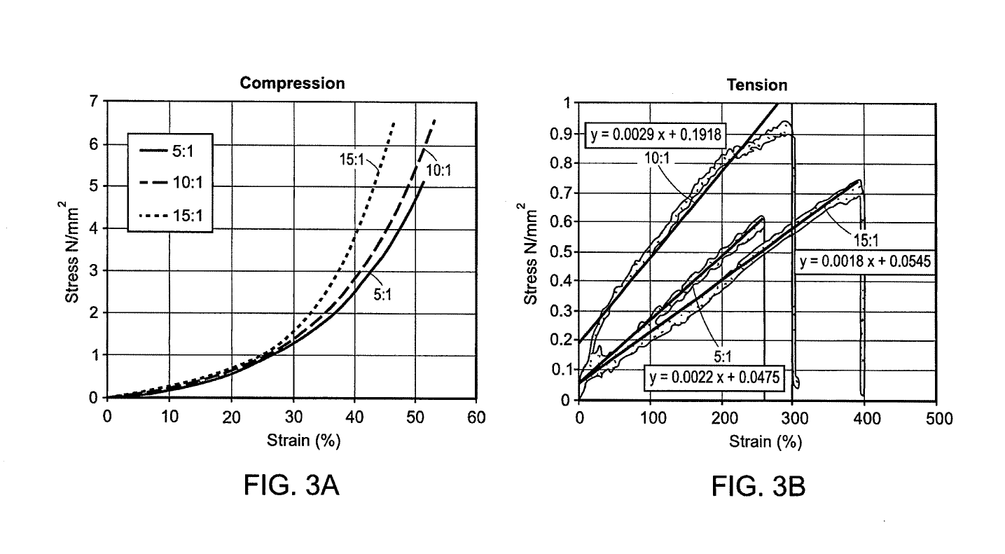

The tear resistance of the material was tested by attaching the sample to one end of a dynamometer. The other end of the dynamometer was kept stationary. A shearing force was applied and gradually incremented. The peak reading of the dynamometer was recorded before the piece tore. When testing tear strength, a ⅛″ incision perpendicular to the edge surface was made and the same shearing three was applied and incremented. Five samples of each material were used and the results were averaged and are shown in FIG. 4. As shown, Sylgard® 186 demonstrated greater tear resistance using both the intact and the slit sample.

3. Determination of Sterilization Resiliency

Samples of Sylgard® 186 were subjected to ten cycles of heat sterilization by autoclaving. It was determined that the tensile and compressive properties of Sylgard® 186 exhibited a 9.3% decrease in elastic modulus after ten sterilization cycles. Therefore, the elastic modulus remains within in an appropriate range for use in the method of the invention of less than 10%.

### EXAMPLE 3

### Porcine Testing Results Comparing Laparotomy Packs to a Barrier

Bowel Packing Time

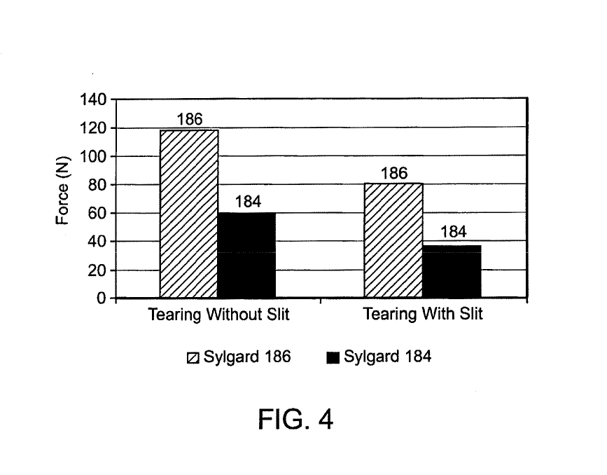

The time required for application of the barrier as well as its effectiveness in retaining the bowels versus the currently used cotton laparotomy packs was quantified in the porcine animal model. Two pigs were used to test the barrier's effectiveness. Before applying the barrier, the surgeon was given only a brief explanation of how the barrier was to be oriented in the cavity and that bowels were to be packed cephally. Once the pigs were fully anesthetized, a ventral midline incision was made to expose the abdominal contents. The incision was retracted using surgical retractor blades. The time required to apply the barrier and the cotton laparotomy packs each were recorded for a series of trials of bowel packing with the barrier or cotton laparotomy packs. As shown in FIGS. 5 A and 6B, packing time was significantly decreased through both an 18 cm and a 12 cm incision (80% and 43% respectively). This demonstrates that use of the barrier can reduce surgical times when bowel packing is required. In addition, the barrier can be removed by inserting one finger in the notch and lifting upward, eliminating the time required to remove individual laparatomy packs as well as eliminating the risk of a retained laparatomy pack.

Maintenance of Intra-Abdominal Temperature

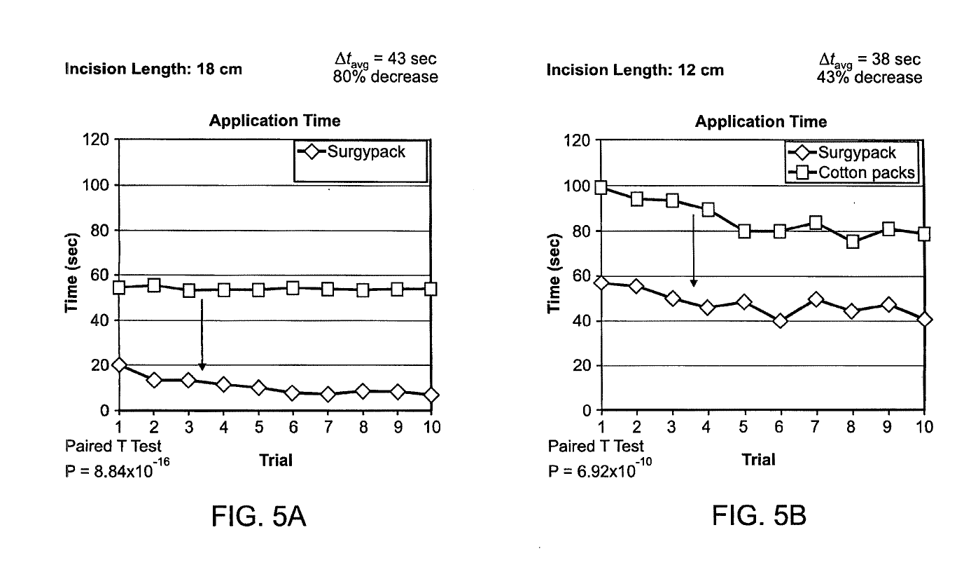

Bowel packing was performed using a barrier or laparotomy packs through a 12 or 18 cm incision. Intra-abdominal temperature was measured upon the completion of bowel packing at six times at 10 minute intervals thereafter. Results are shown in FIG. 6. During the course of the experiment, cavity temperature decreased in the pig in which abdominal packing was performed with laparotomy pads, resulting in an abdominal cavity temperature about 7° C. below body temperature at the end of the experiment. Conversely, bowel packing with the barrier resulted in an increase in abdominal cavity temperature over the course of the experiment bringing cavity temperature to about 4.5 degrees below body temperature after 40 minutes of bowel packing with the barrier n=1).

Maintenance of Bowel Retention

Bowel packing was performed using a barrier or laparotomy packs through a 12 or 18 cm incision. Bowel retention was observed at 5 minute intervals for a total of one hour under both static and dynamic conditions. The static phase of the test was conducted by leaving the packed bowels in the fully anesthetized animal, undisturbed by the surgeon. The barrier is only subject to the forces produced, by the animal's physiological processes.

The dynamic phase of the test began with the surgeon performing a nephrectomy. Attempts to dislodge the barrier included, grasping the animal by the pelvis and shaking the abdomen.

Bowels are considered to have entered the surgical field (i.e., no longer be retained) once a segment greater than 1 cm in length breaks the plane formed by extending the surface of the barrier nearest the surgical field.

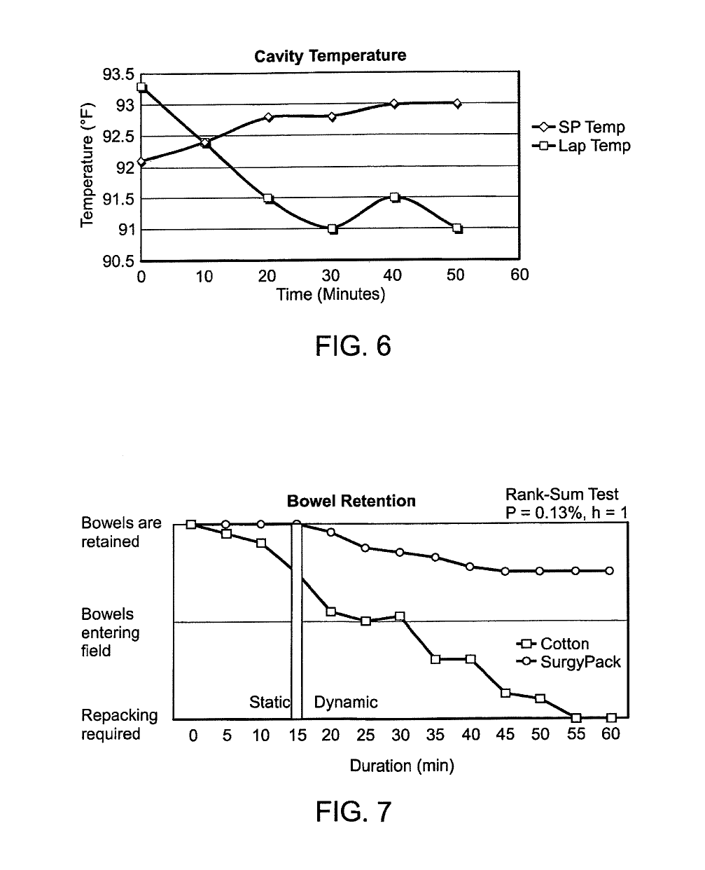

The barrier was found to retain the bowels throughout the course of the experiment, whereas the significant bowel leakage occurred with the laparotomy packs after as little as 15 minutes with bowels entering the field at 20 minutes (FIG. 7). After less than one hour, repacking was required after packing with laparotomy packs. These data demonstrate that the barrier provides better bowel retention than standardly used laparotomy packs.

An embodiment of the present invention also includes a barrier for bowel retention and method of use, wherein the barrier is sufficiently collapsible that the barrier may be inserted into an abdomen via a laproscopic surgery procedure. Hereinafter, such a barrier will be referred to, for convenience, as a collapsible barrier. By way of example, the collapsible barrier may have general dimensions and configuration similar to or the same as those detailed above, but also having sufficient collapsibility to be inserted into the abdominal cavity through a trocar or trocar-like medical barrier or through a relatively tiny incision. This as compared to the incision typically made through the ventral side of the patient, which may result after retraction of the skin in an opening about 15 inches in diameter or more.

The collapsible barrier may be collapsed rolled, folded or otherwise bunched. together) to fit into the cannula of the trocar, etc. Sufficient force applied to the collapsible barrier causes the barrier to move through the cannula of the trocar and into the abdominal cavity. Once in the abdominal cavity, the barrier is uncollapsed (e.g., unrolled, unfolded, unbunched, etc.) to expand to the configuration(s) detailed herein. The barrier has sufficient structural rigidity after it is uncollapsed such that it maintains the bowels in a retained state in a manner having the same or about the same efficacy as the barrier detailed above.

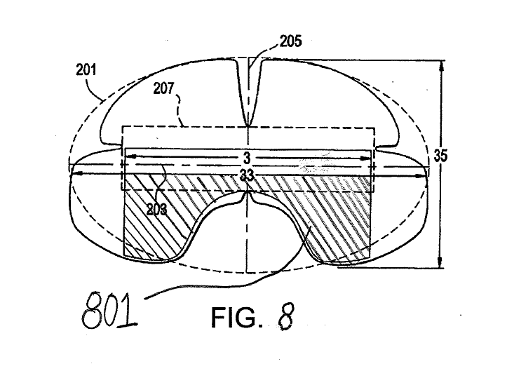

In an exemplary embodiment, such collapsibility may be achieved by making a barrier having a thickness less than the thicknesses of the barrier detailed above with respect to FIGS. 1-7. By way of example, with respect to FIG. 1E, the thickness of the material in the central region of the barrier may be less than and/or the amount of material in those areas may be less than that of the barrier detailed above with respect to FIGS. 1-7, as will now be described in greater detail. By central region, it is meant, for example, the area encompassed by box 207 and immediately below box 207 (i.e., downward with respect to the page orientation) of the collapsible barrier and/or the shaded area 801 of FIG. 8

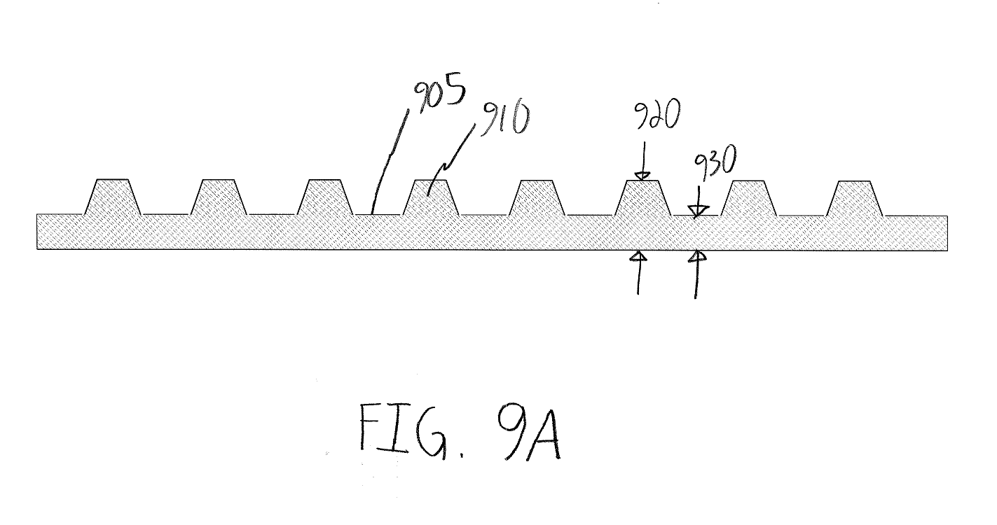

In an exemplary embodiment, the portion of the barrier material in the central region may have a greater global thickness than that of the collapsible barrier at the perimeter. However, locally, the thickness varies such that the thickness is about the same as the thickness at the perimeter. Referring to FIG. 9A, there is depicted a cross-sectional view taken along line 205 of FIG. 8, where the thickness of the material in the central region has localized reductions in thickness. Specifically, the thickness of the material at this section varies due to the protrusions 910 separated by valleys 905 as shown, with thickness 920 being about 0.25 to about 0.5 or about 0.75 inches, and thickness 930 being about 0.05 to about 0.1 or about 0.2 or about 0.3 inches, etc. FIG. 9B depicts a cross-sectional view taken along line 205 of FIG. 8, where the thickness of the material at the shaded area 801 varies due to the protrusions. As will be seen in FIG. 9B, there is an extended area of reduced thickness 940 in the area above (with respect to the page) the shaded area 801 of FIG. 8, whereas in the embodiment of FIG. 9A, that area includes protrusions.

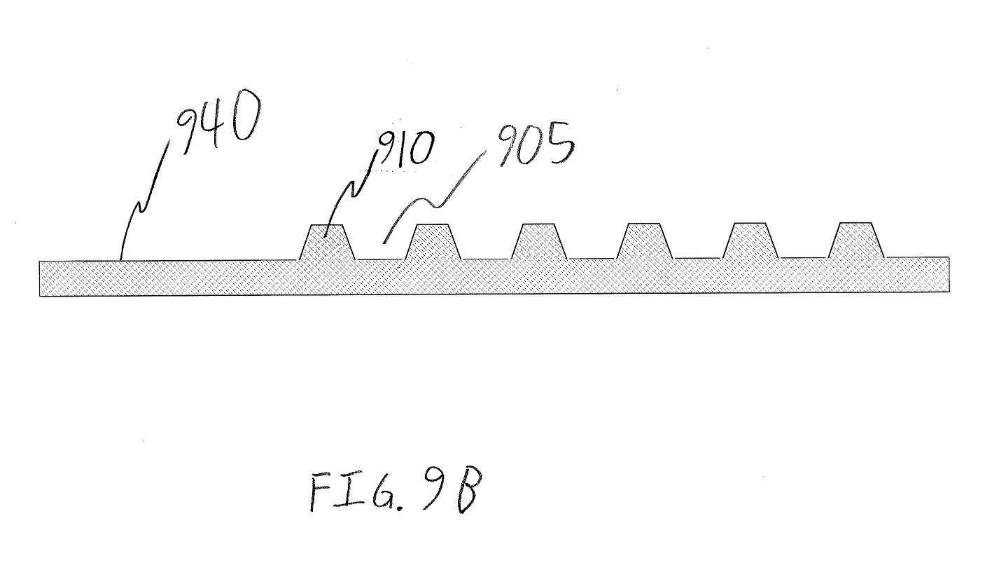

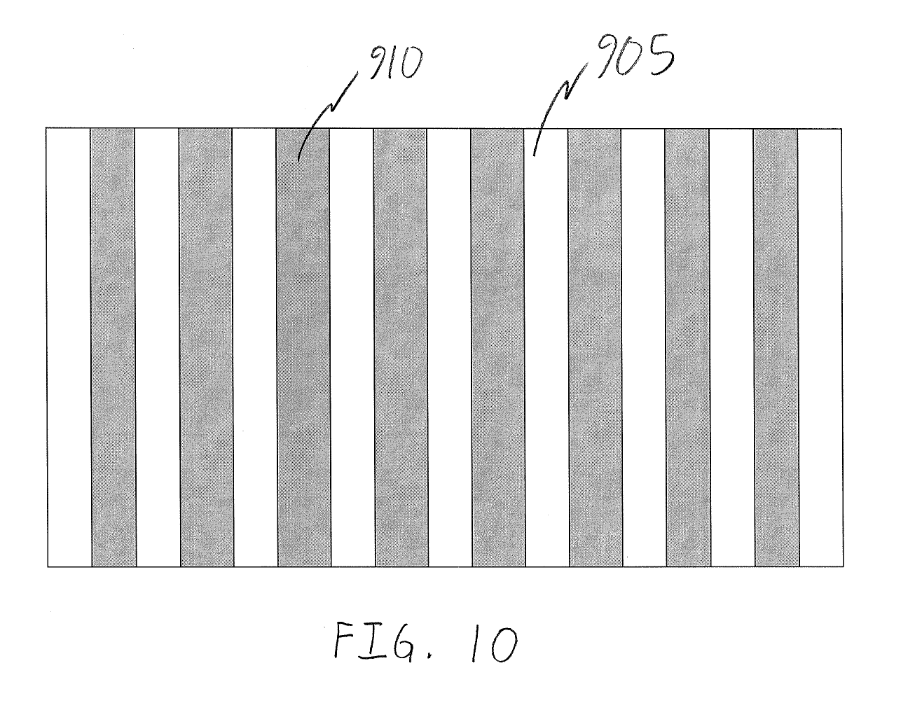

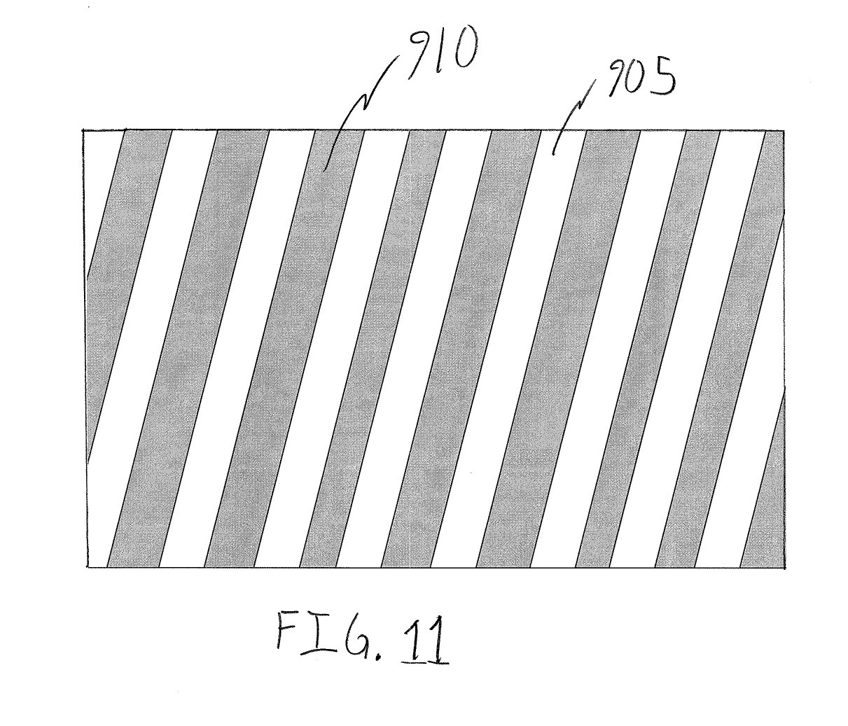

The protrusions 910 may be formed parallel to one another, as shown in FIG. 10, which is a view providing additional details of the area within box 207 of FIG. 1E (the general configuration is applicable to the embodiment of FIG. 9B, where the protrusions would not extend the full vertical distance, as would be understood). The alignment of the protrusions 910 may be canted, as shown in FIG. 11, which is a view providing alternate details of the area within box 207 of FIG. 1E (the general configuration is applicable to the embodiment of FIG. 9B, as would be understood). Alternatively, the protrusions may be aligned at an angle from that depicted in FIG. 10 (e.g., approximately 90 degrees). In another embodiment, the protrusions may be in the form of checkered protrusions, as shown in FIG. 12, which is a view providing alternate details of the area within box 207 of FIG. 1E (the general configuration is applicable to the embodiment of FIG. 9B, as would be understood). In another embodiment, the protrusions may be in the form of discrete unconnected protrusions, as shown in FIG. 12, which is a view providing alternate details of the area within box 207 of FIG. 1E (the general configuration is applicable to the embodiment of FIG. 9B, as would be understood).

It is noted that while the protrusions 910 are depicted on the convex side of the collapsible barrier (away from the bowels when inserted into the abdominal cavity), the protrusions 910 may be located on the concave side of the collapsible barrier, and/or on both sides of the collapsible barrier.

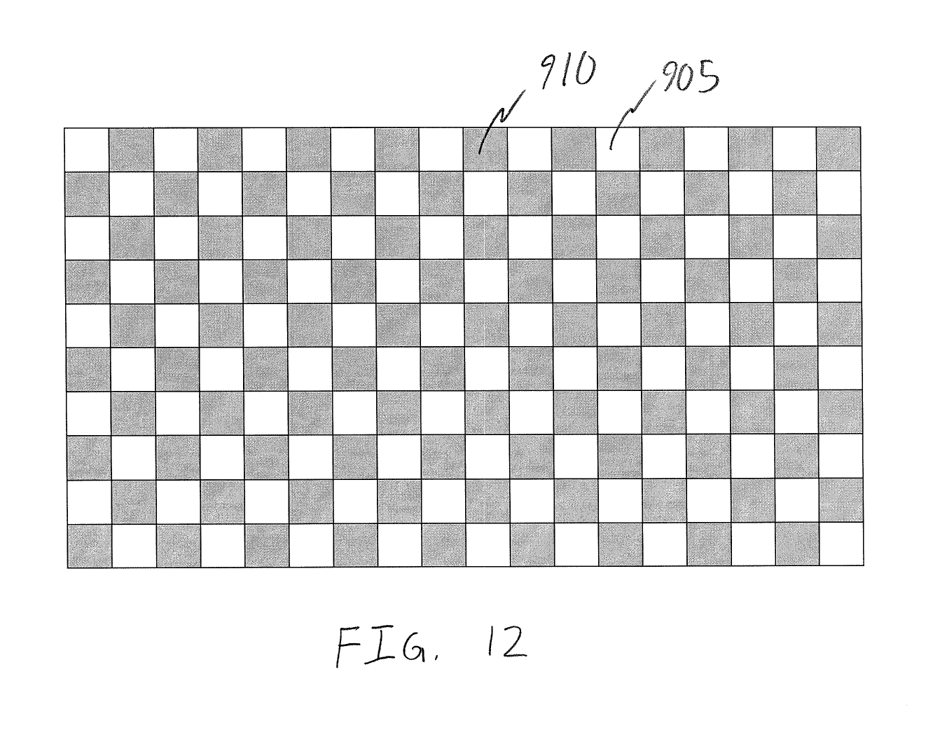

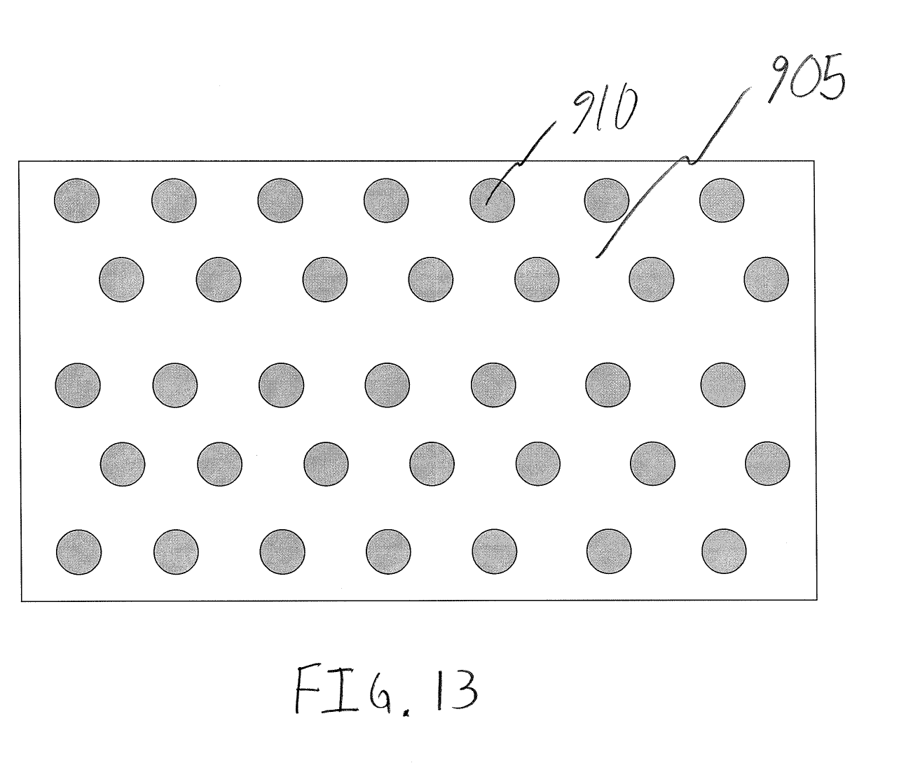

FIG. 14 depicts a cross-sectional view taken through an area of the collapsible barrier when the collapsible barrier is in a collapsed position. As may be seen, the protrusions 910 fit into the valleys 905, thus enhancing the collapsibility of the collapsible barrier the barrier may be collapsed into a smaller volume). An embodiment of the present invention includes a method. of collapsing the collapsible barrier such that the protrusions 910 fit as shown in FIG. 13.

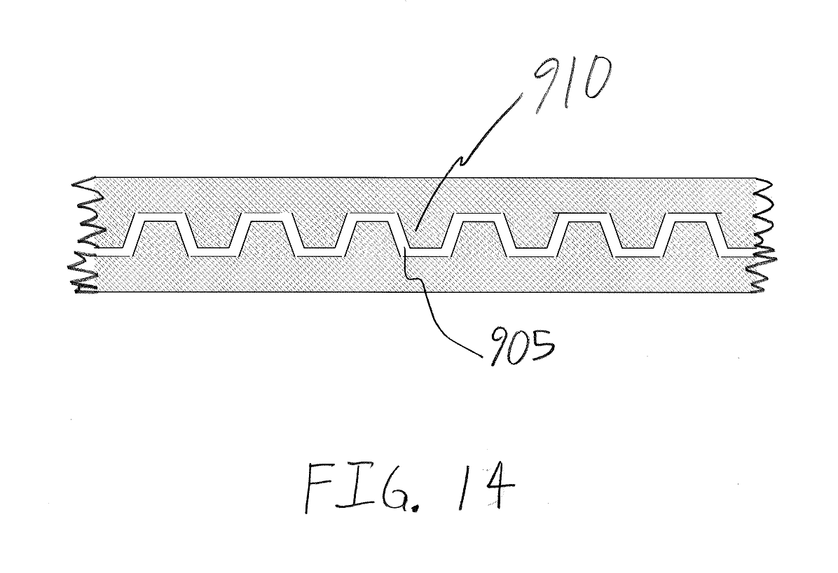

FIG. 15 depicts another embodiment where the protrusions may be in the form of a waffle pattern, as shown. FIG. 15 is a view providing alternate details of the central portion of the barrier (the general configuration is applicable to the embodiment of FIG. 9B, as would be understood). As will be understood, this embodiment may not be as readily collapsible as some of the other embodiments where the protrusions fit into valleys as shown in FIG. 14. However, such an embodiment reduces the amount of material utilized to make the barrier/the collapsible barrier, thus reducing costs (e.g., cure times are reduced). In this regard, any of the embodiments of FIGS. 9-15 may be used in barriers simply to reduce manufacturing costs, regardless of whether or not enhanced collapsibility results therefrom. Further, such configurations permit an adequate buffer to be placed between the retractor blades and the internal organs/bowels reducing the amount of material used to manufacture the barrier. Such embodiments may result in a decrease in the structural rigidity of the barrier as compared to the solid/thick sections detailed above with respect to the embodiments described above with respect to FIGS. 1-7.

Accordingly, with respect to the embodiments of FIGS. 9A-15, there is a collapsible barrier including a central region that, has localized areas where, relative to one another, the thicknesses of these areas is substantially different. The central region has localized areas of thick and thin sections, relative to one another.

An embodiment of the collapsible barrier utilizes fluid channels, which may be imbedded in the material of the collapsible barrier. These fluid channels are configured to receive fluid when the collapsible barrier is in a collapsed state. The introduction of fluid into the channels uncollapses the collapsible barrier, expanding it to dimensions sufficient to retain interior organs/bowels. In an exemplary embodiment, the collapsed collapsible barrier is inserted into the abdominal cavity via the cannula of the trocar, and a flexible tube extends from the collapsible barrier through the cannula and out of the patient to a fluid pump. The fluid pump is activated by a surgeon or a nurse or other type of technician, and the fluid pump pumps fluid through the flexible tube and into the fluid channels of the collapsible barrier, thereby causing the collapsible barrier to uncollapse. In an exemplary embodiment, the fluid introduced into the fluid channels “inflates” the collapsible barrier, thereby providing rigidity to the collapsible barrier.

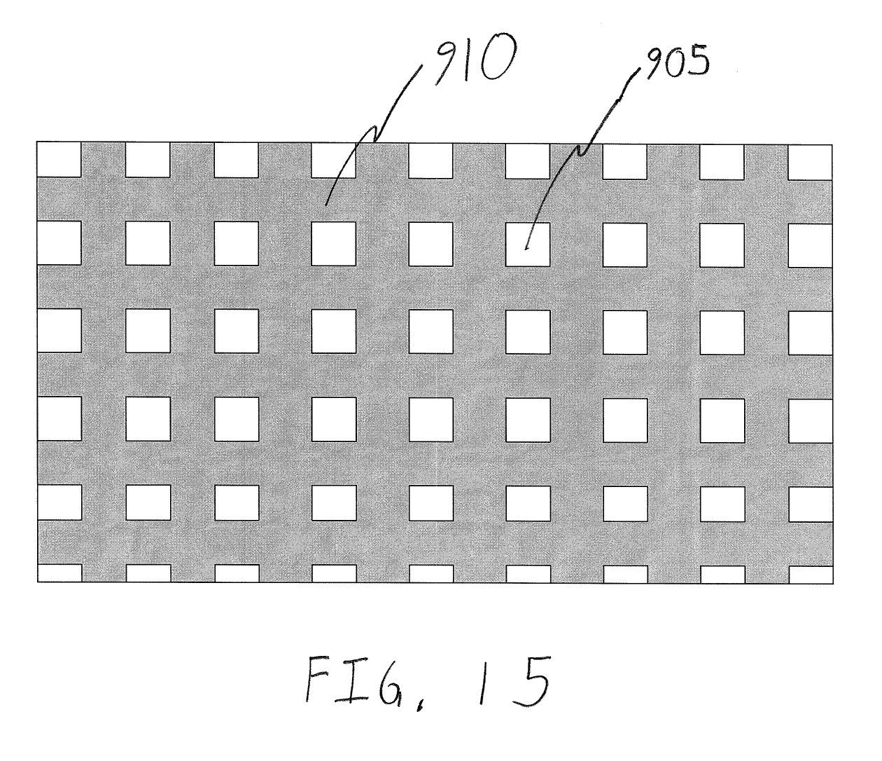

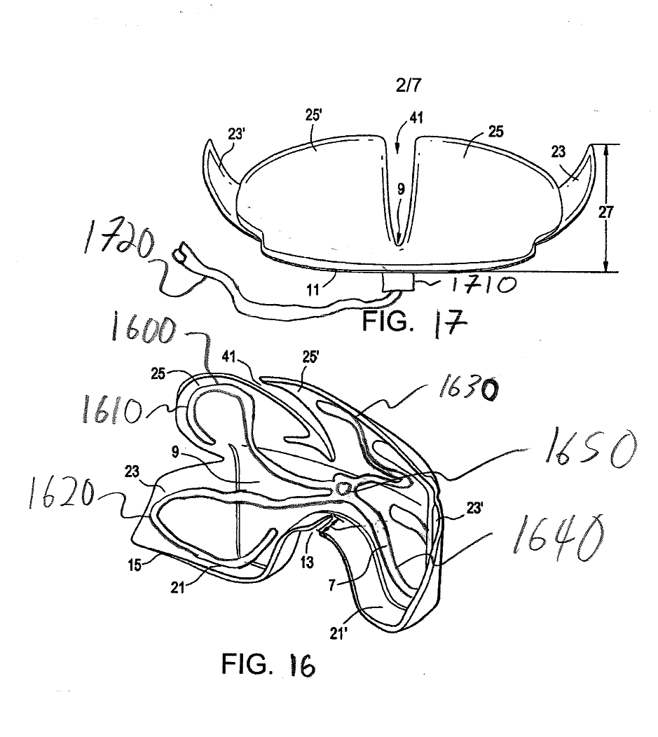

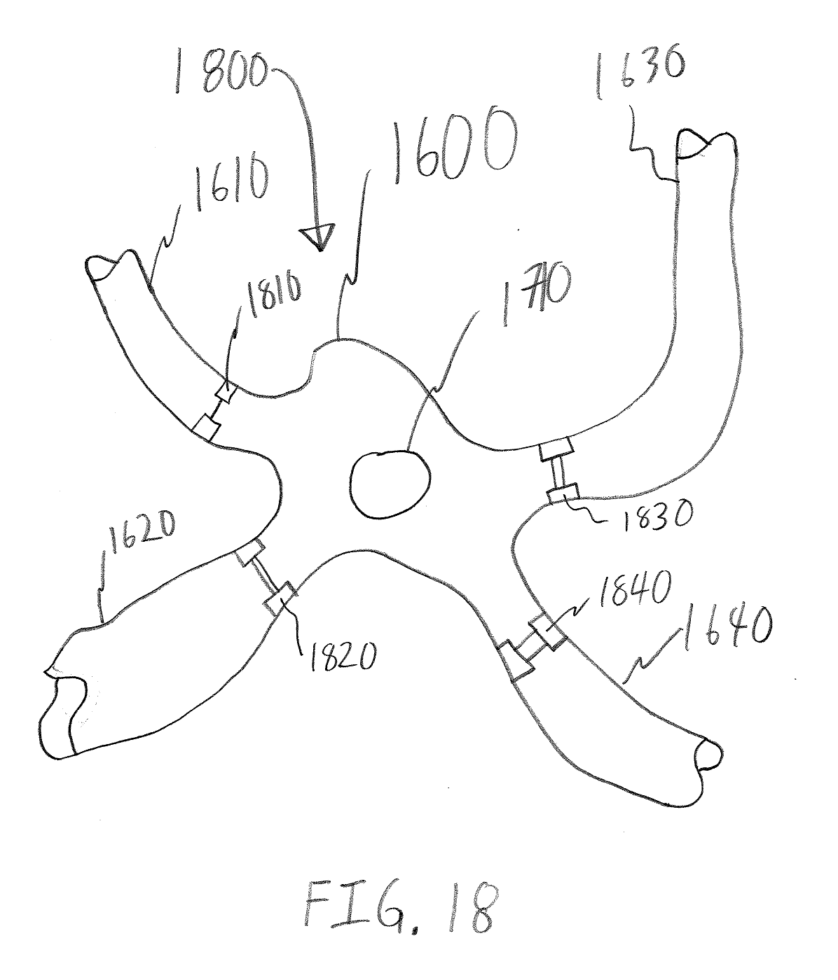

An embodiment of the present invention utilizes a fluid channel network, which may include conduits and valves, such that the collapsible barrier uncollapses a section at a time. By way of example, referring to FIGS. 16 and 17, there is a fluid channel network 1600 including fluid channel branches 1610, 1620, 1630 and 1640 extending, respectively, into sections 25, 23, 25′ and 23′ of the collapsible barrier. The fluid channel network 1600 is in fluid communication with connector 1710, which is in turn in fluid communication with flexible tube 1720. FIG. 18 depicts a schematic of a valve assembly 1800 usable in the fluid channel network 1600 to control fluid entry into the fluid channel branches. As may be seen, valve assembly 1800 includes valves 1810, 1820, 1830 and 1840 respectively positioned in the various fluid channel branches.

The valves of this embodiment comprise pressure sensitive valves in the form of rupturing diaphragms that rupture upon the application of sufficient fluid pressure. In the embodiment depicted in FIG. 18, the valves are configured to rupture in sequence, valve 1810 rupturing at a first pressure, valve 1820 rupturing at a second pressure greater than the first pressure, 1830 rupturing at a third pressure greater than the second pressure, valve 1840 rupturing at a fourth pressure greater than the third pressure. In the embodiment depicted in FIG. 18, this is achieved by having a diaphragm wall thickness that respectively increases with each valve 1810-1840, as is depicted. To uncompress the compressible barrier, a fluid is introduced through connector 1710 into the network 1600. The pressure of the fluid increases to the first pressure, at which point valve 1810 ruptures, permitting fluid to enter the fluid channel branch 1610, thereby uncompressing section 25. The pressure of the fluid then increases to the second pressure, at which point valve 1820 ruptures, permitting fluid to enter the fluid channel branch 1620, thereby uncompressing section 23. The pressure of the fluid then increases to the third pressure, at which point valve 1830 ruptures, permitting fluid to enter the fluid channel branch 1630, thereby uncompressing section 25′. The pressure of the fluid then increases to the fourth pressure, at which point valve 1840 ruptures, permitting fluid to enter the fluid channel branch 1640, thereby uncompressing section 23′.

In other embodiments, other valving configurations may be used (the valves may be electromechanical valves, etc.). By sequencing the operation of the valves, various sections of the collapsible barrier may be uncollapsed in a desired sequence.

It is noted that in an exemplary embodiment, the collapsible barrier is collapsed in a manner to avoid kinking the material/creasing the material in such a way that the material substantially plastically deforms, thereby imparting a permanent kink/crease that interferes with the use of the collapsible barrier. That is, an embodiment of the present invention entails collapsing the barrier and/or making the barrier from material such that deformation of the barrier during and/or upon collapsation is substantially elastic.

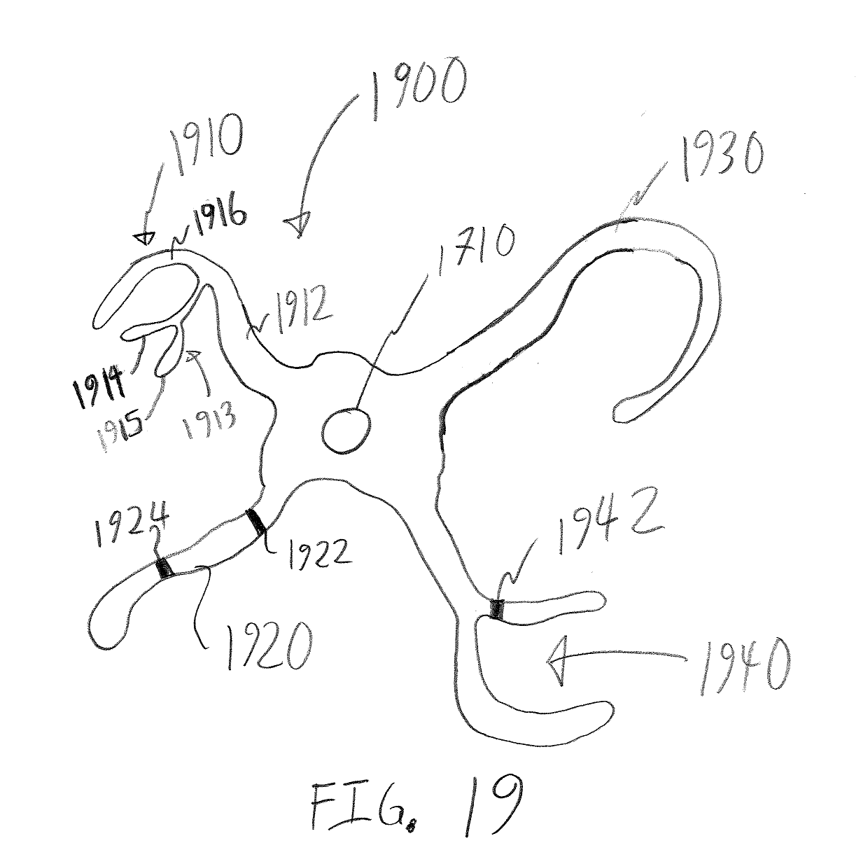

It is noted that while the embodiments of FIGS. 16-18 depict four fluid channel branches (one for each section 23, 25, 23′, 25′), other embodiments may include additional channel branches and/or sub-branches to those branches, which may or may not be separately valved. In this regard, FIG. 19 depicts an alternate embodiment of a fluid channel network 1900. Fluid channel branch 1910, which positionally and functionally corresponds to branch 1610 described above, includes main branch 1912, and sub-branches 1916 and 1913. Sub-branch 1913 includes sub-branches 1914 and 1915. The diameters of these branches may be various configured to control the flow of fluid therein, thereby controlling uncollapsing of the barrier. In an embodiment, branch 1910 does not include a valve, and thus branch 1910, and, along with branch 1930 (which also does not include a valve, expand before branches 1920 and 1940. Branches 1920, 1930 and 1940 positionally and functionally correspond to branches 1620, 1630 and 1640 described above.

As may be seen, branch 1920 includes valves 1922 and 1924, thereby micro-controlling the expansion of the associated section of the barrier. In an embodiment, the valve 1922 is configured to open at a given pressure, but valve 1924 is configured to open at a higher pressure. In an embodiment, a valve in another branch may be configured to open at a pressure lower than the opening pressure of valve 1924, thereby synchronizing the uncollapsing of sub-sections with the uncollapsing of other sections and/or sub-sections. Valve 1942 may be used in conjunction with valve 1924 and or valve 1922 in such a manner.

The fluid channel network may be used, in some embodiments, to provide warmth to the internal organs/bowels of the patient. In an embodiment, the fluid inserted into the fluid channel network may be heated, thereby transferring heat to the patient.

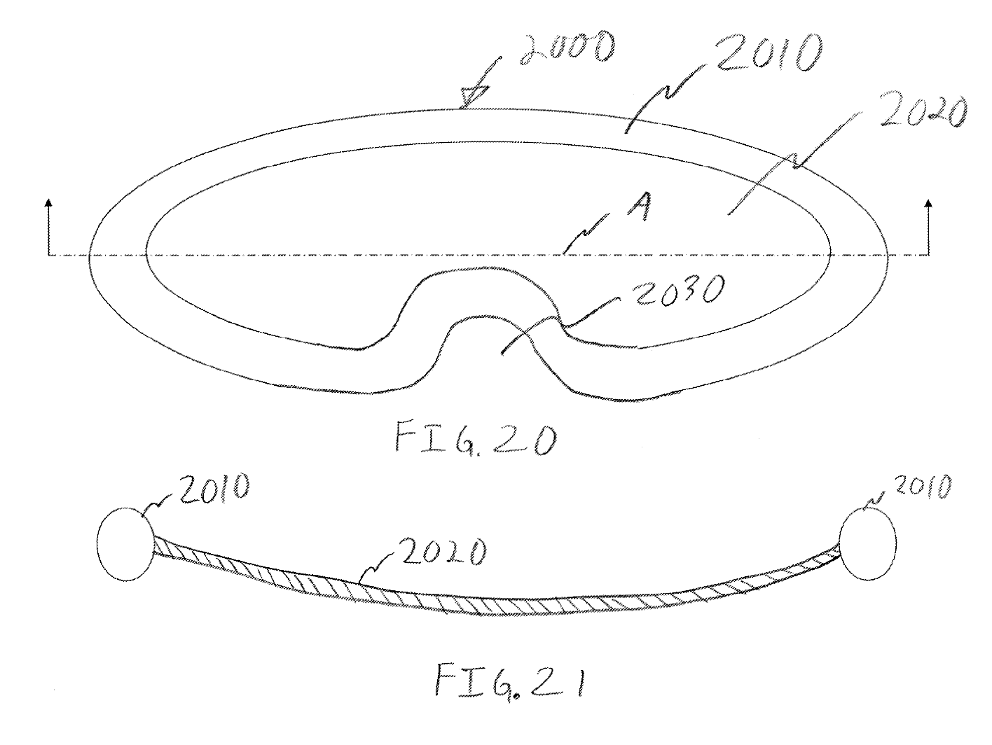

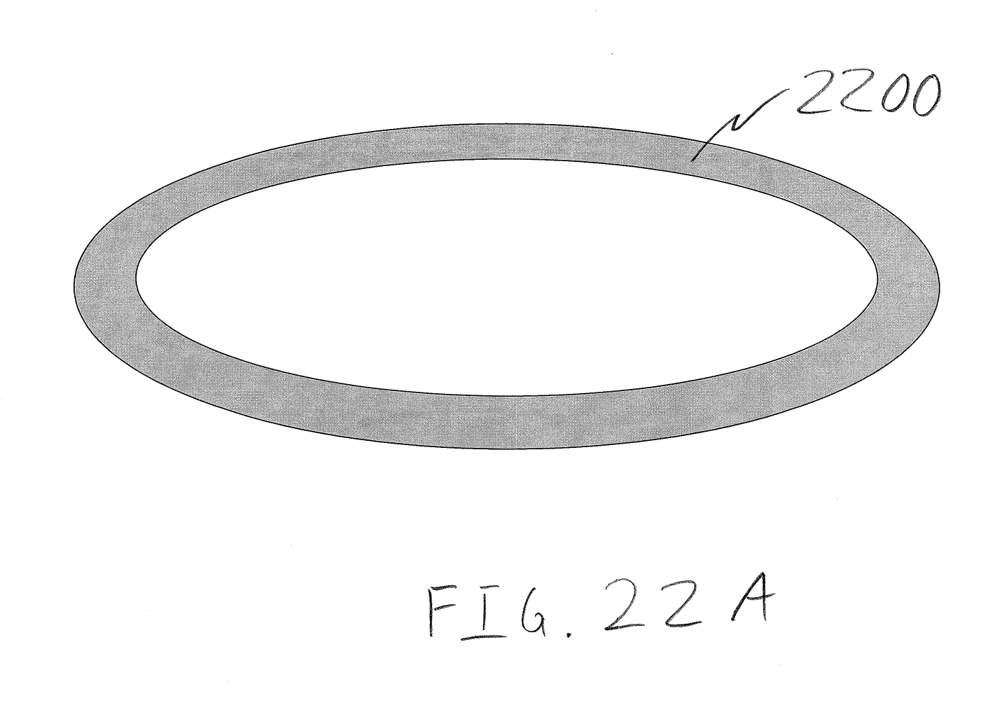

FIG. 20 depicts another exemplary embodiment of the present invention in a collapsible barrier form of a diaphragm assembly 2000 that may be inserted into the abdominal cavity. FIG. 20 details that diaphragm assembly 2000 includes an outer frame 2010 and an interior diaphragm 2020 (which may be a plastic film having substantially uniform thickness) that extends from the frame 2010. FIG. 21 depicts a cross-sectional view of the diaphragm assembly 2000 of FIG. 20. As may be seen, frame 2010 is hollow. In an exemplary embodiment, frame 2010 is a fluid channel that receives fluid in a manner analogous to the fluid channels described above to uncollapse and/or strengthen the diaphragm assembly 2000. As may be seen, there is a gap 2030 in the diaphragm assembly to accommodate the spinal column, akin to element 13 detailed above.

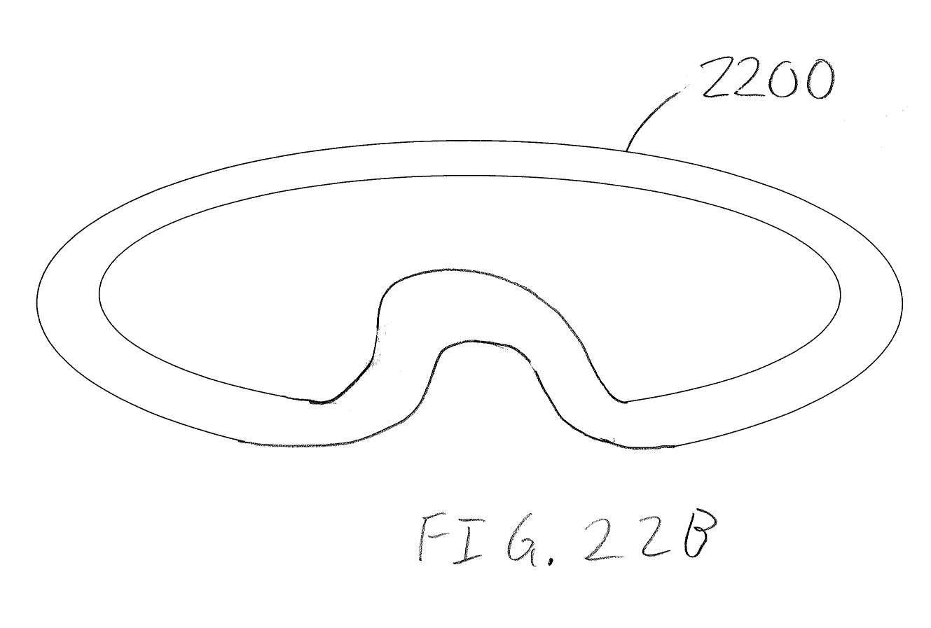

FIG. 22A depicts an alternate embodiment of a diaphragm 2200, where the frame and diaphragm material is sufficiently flexible that it sufficiently contours around the spinal column while establishing a sufficient barrier between the surgical area and the internal organs. Along these lines, the diaphragm assembly 2200 may take the shape as depicted in FIG. 22B to contour around the spinal column. Accordingly, FIG. 22A depicts the diaphragm assembly 2200 in its uncollapsed state, free of constriction or other forces interposed by internal organs/body parts of the patient.

While diaphragm 2020 is depicted as concave, in other embodiments, diaphragm 2020 may be substantially flat.

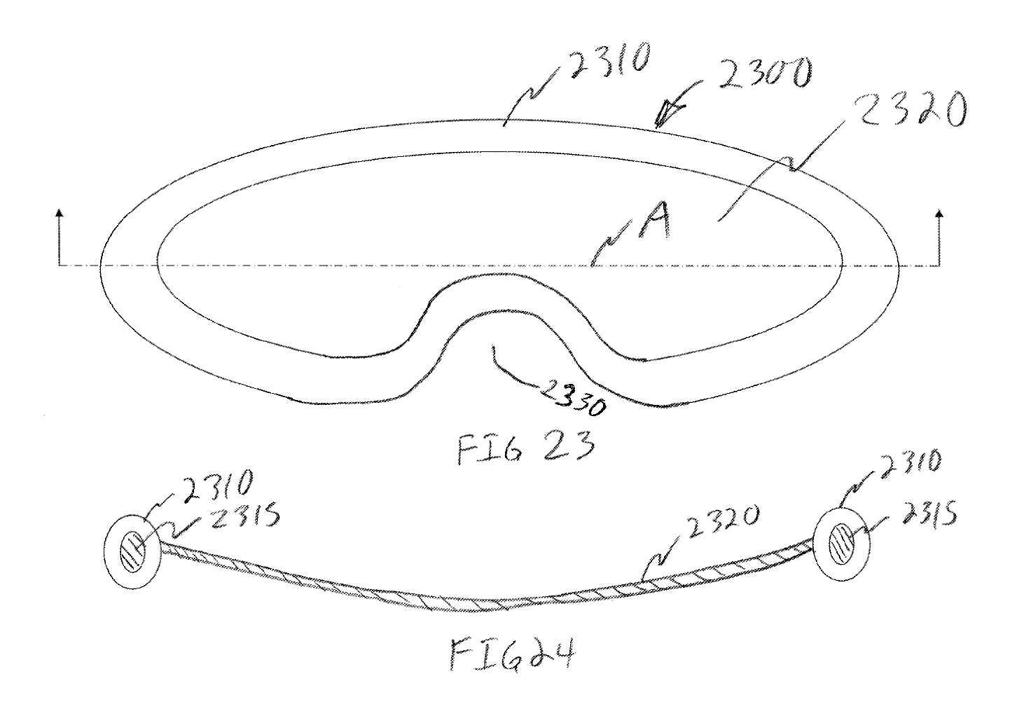

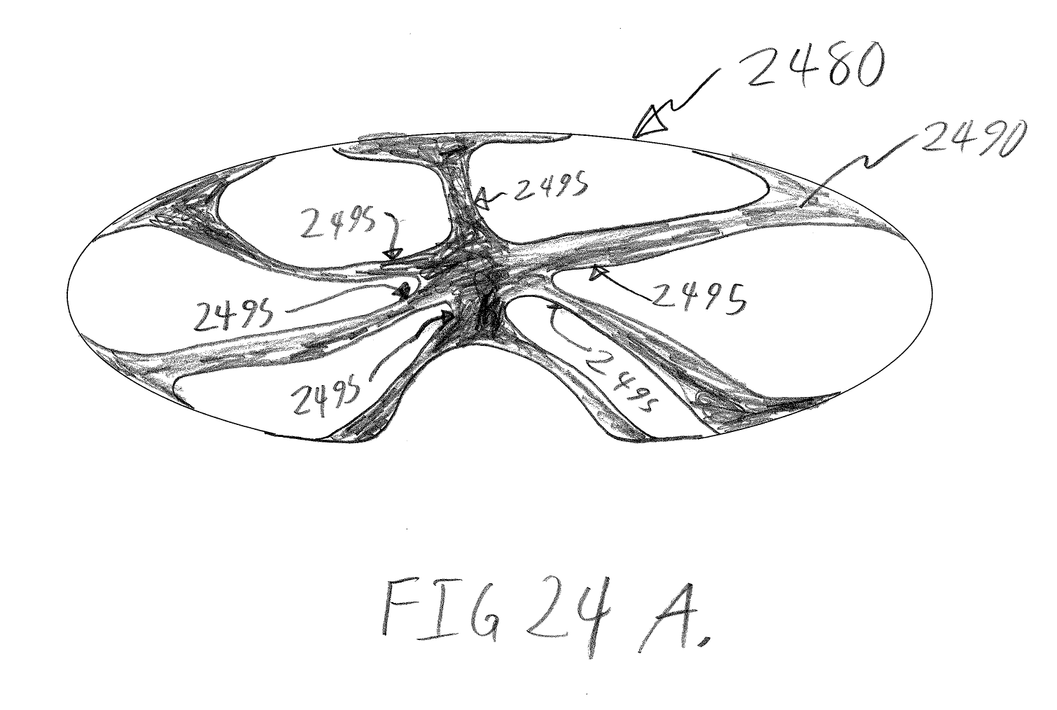

FIG. 23 and FIG. 24 (a cross-sectional view of FIG. 23) depict an alternate embodiment of a diaphragm assembly 2300 corresponding to diaphragm assembly 2100 detailed above. Diaphragm assembly 2300 includes frame 2310 (corresponding to frame 2010) and diaphragm 2320 (corresponding to diaphragm 2020). Frame 2310 includes a skeleton 2315 that provides structural support to the diaphragm but is also sufficiently flexible to permit the diaphragm assembly 2300 to be collapsed and/or to conform to the spinal column without substantially plastically deforming the skeleton 2315. The skeleton 2315 may be made of metal or other flexible materials. Metal wire may be used in some embodiments as skeleton 2315. Elastomer materials may be used to form the skeleton 2315. Skeleton 2315 is depicted as a solid component, but may be in the form of a tube or a composite material it may be a laminate or a bundle (e.g., a cable), etc.)

FIG. 24A presents an alternate embodiment of a diaphragm assembly, 2480, with a plurality of fluid channels 2490 (shaded areas of FIG. 24A). These channels 2490 may be formed on a film making up the diaphragm. Valves 2495 may be located as shown to operate as detailed herein, with a connector 1710 located approximately in the geometric center of the assembly. The fluid channels may be in the form of a star pattern. An embodiment may include the combination of the frame of FIG. 20 and the pattern of FIG. 24A.

In an embodiment of the present invention, the outer boundary of the diaphragm assemblies expand to press against the interior of the abdominal cavity (e.g., to form an interference fit), thus holding the diaphragm assembly in place.

The diaphragm assemblies described herein may be collapsed by folding or twisting the diaphragm upon itself to a sufficient collapsibility level that the diaphragm may be introduced through the cannula of a trocar in a minimally invasive surgical procedure. In an exemplary embodiment, the diaphragm assemblies are of configurations such that, with respect to FIGS. 20 and 24, the left (or right) side of the diaphragm assemblies may be twisted 180 degrees about the axis A, and then the left (or right) side may be folded onto the opposite side. Further twisting and/or folding may be utilized to further collapse the diaphragm assemblies.

In an embodiment of the present invention, insertion of the collapsible barriers described herein may be accomplished by holding a portion of the collapsed collapsible barrier with a forceps and inserting the collapsible barrier through a minimally invasive incision/cannula of a trocar and into the abdominal cavity.

Removal of the collapsible barrier may be accomplished by pulling the collapsible barrier back through the minimally invasive incision/cannula of a trocar. In an embodiment of the present invention, the fluid used to fill the fluid channels may be evacuated. This may be accomplished by applying a vacuum to the fluid channel network to suck the fluid out of the channels or by incorporating a valve that opens to permit the fluid to be evacuated (e.g., deflating the fluid channel network). In an exemplary embodiment, referring to the embodiment of FIGS. 16-18, a fifth valve (not shown) is added to the fluid channel network 1800. The fifth valve is rupturable at a pressure greater than the fourth pressure (which ruptures valve 1840). This fifth valve is provided at an interface between the fluid channel network and the exterior of the collapsible barrier (which may be open to the atmosphere and/or open to the interior of the patient). Upon opening (rupture) of the valve, the fluid is free to flow out of the network, thus permitting the collapsible barrier to be more-easily withdrawn from the abdominal cavity.

It is noted that in an exemplary embodiment, the fluid used to fill the fluid channels is a saline solution or carbon dioxide gas or other fluid which is biocompatible with a patient. Thus, permitting fluid from the fifth valve to safely intermingle with body fluids of the patient. Magna-rheological fluid and/or electro-rheological fluid may be used in the fluid channels. This will provide a magnetic field and/or an electric current, respectively.

Once the barrier is no longer needed in the abdomen, the barrier may be recollapsed so that it may be withdrawn from the abdomen through the cannula of the trocar and/or through the incision in the abdomen.

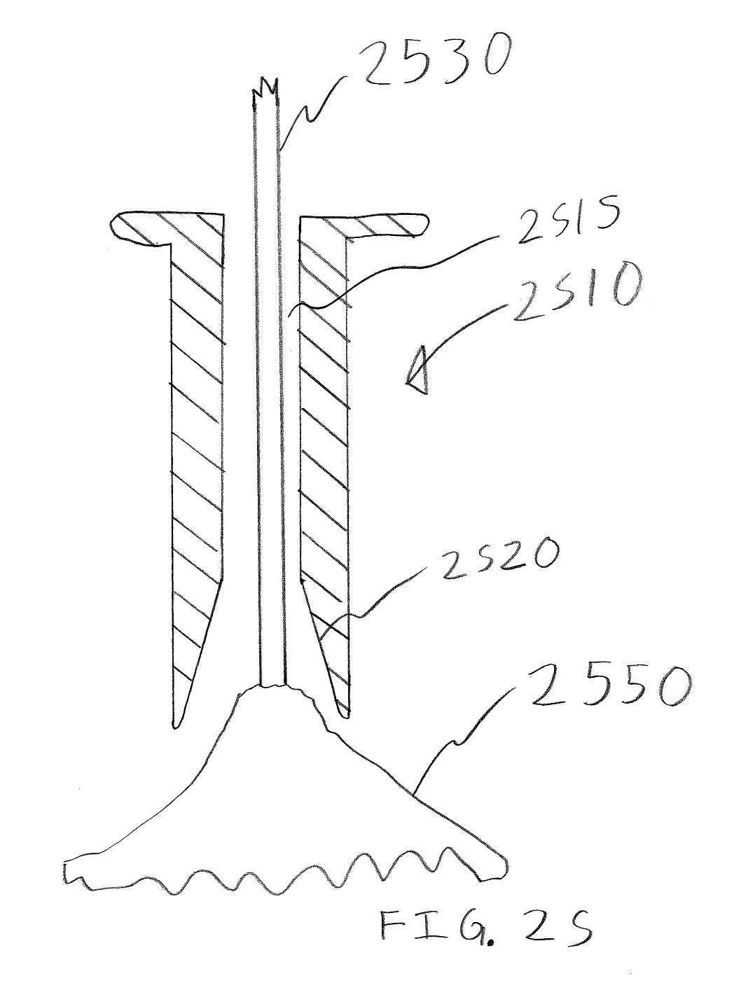

In an exemplary embodiment, a funnel barrier is used to aid in the removal of the collapsible barrier. FIG. 25 depicts a cross-sectional view of a trocar 2510 including a funnel-section 2520 used to assist in the removal of collapsible barrier 2550 (where collapsible barrier 2550 may correspond to any of the collapsible barriers detailed herein and variations thereof). Removal component 2530 is connected or otherwise attached to collapsible barrier 2550. Removal component 2530 may correspond to the flexible tube used to pass fluid into the collapsible barrier 2550, or may be a separate removal cord or may correspond to a pair of forceps. As may be seen, funnel section 2520 guides collapsible barrier 2550 into the cannula 2515. In the embodiment depicted in FIG. 25, the diameter of the cannula 2510 may be about 1 inch to about 1.5 to about 2 inches.

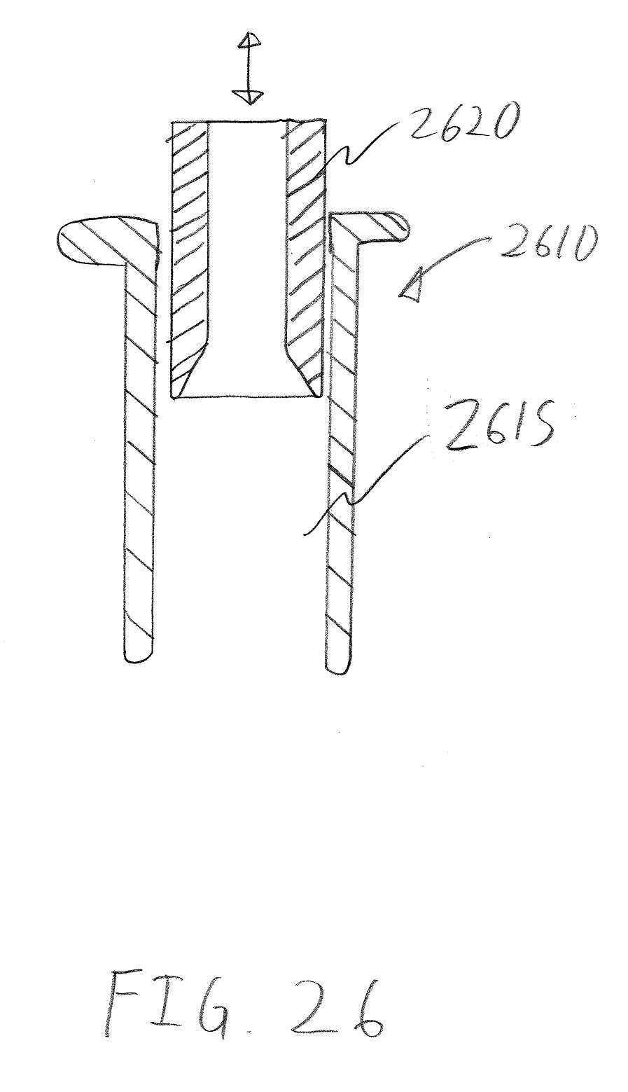

FIG. 26 depicts an alternate embodiment utilizing a movable funnel component. Specifically, funnel 2620 is movable (slidable) down the cannula 2615 of trocar 2610 so that it guides the collapsible barrier 2550 (not shown) into the cannula 2615 of trocar 2610. The funnel 2610 may interface with the trocar 2610 such that it will not extent too far out of the cannula 2615. In an exemplary embodiment, the funnel 2610 is configured with a barrier to lock or otherwise secure the funnel 2610 in place so that the funnel will not be pulled back through the cannula with the collapsible barrier 2550.

An exemplary embodiment of the barriers described herein, including the collapsible barrier, includes a barrier having a blood absorber. In an exemplary embodiment, capillary tubes or other tubes/gaps are formed in the surface of the barrier so that blood and other body fluids are wicked up into or otherwise absorbed into the barrier. In an exemplary embodiment, the surface is somewhat roughened. The degree of roughness is such that it will not cause abrasions or the like to the organs of the patient, but has a blood/body fluid absorbing feature (e.g., blood may wick into the roughened sections. In an exemplary embodiment, these feature (capillary tubes (the openings) rough section, absorbable foam material, etc.) may be located at section 207 and the area immediately below that section with respect to FIG. 1E and/or at section 801 with respect to FIG. 8.

It is noted that the barriers described herein and variations thereof may be used to re-position organs (bowels and other organs) during surgery, and the collapsible barriers may be used to reposition organs in laproscopic surgery.

It is noted that the fluid channels may be attached to the base materials of the collapsible barriers, or may be molded as an integral part of the barriers.

Embodiments of the present invention may combine the barrier technologies described herein with balloon technologies. In an embodiment, a balloon attached to a barrier (which may be integral with the barrier) may be filled with saline or other fluids to stiffen the barrier after insertion into the abdominal cavity.

Embodiments of the barrier (including the collapsible barrier) may be made from thermoplastic elastomers, such as by way of example, styrenic block copolymers, polyolefin blends, elastomeric TPU, thermoplastic coployester, and thermoplastic polyamides, polysulfide rubber, and/or thermoplastic vulcanizates. Still further, thermoset elastomers, including polyisoprene, may be used to make the barrier. Saturated rubbers may also be used, such as, for example, EMI and EPDM, Epichlorohydrin rubber, polyacrylic rubber, florosilicone rubber, fluoroelastomers, perfluoroelastomers, polyether block amides, cholosulfonated. polyethylene, ethylene-vinyl acetate. Non-elastomeric polymers may also be used to make the barrier, including PTFE, PLT, PTE, LIVE, Cross-linked PE, HDPE, PE, Polypropylene, PEEK, PVC, polycarbonate, Polystyrene, and/or PEI. Composite materials may also be used, which may include the above-mentioned polymers and materials combined with reinforcing fibers, fillers, woven materials, polymer foam inserts, etc.

Polymers with relatively low Tg/softening points that would deform with steam sterilization may be used to manufacture the collapsible barrier. An embodiment of the present invention includes features/the use of materials that reduce the likelihood that the barrier may be reused, thus reducing the spread. of disease (e.g., AIDS).

An embodiment of the present invention includes scoring the thicker sections of the barrier after the barrier is molded. Such scoring may provide thin channels in the barrier.

Computer chips/microchips may be included into the barriers to permit RFID evaluation (counting, supply management, source control, etc). A temperature sensitive dye may be incorporated into the barriers to indicate whether the barrier has been steam sterilized. Sensors may be included in the collapsible barrier to sense temperature, humidity, pressure, etc.

PET potential materials, such as those used in PET medical balloons, may be used to form some barriers. Non-extensible nylon or biocompatible polyimide-potential materials may also be used.

The invention described and/or claimed herein is not to be limited in scope by the specific preferred embodiments herein disclosed, since these embodiments are intended as illustrations, and not limitations, of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

What is claimed is:

1. A device for human organ packing in a laproscopic surgical procedure, comprising:
an expandable barrier configured to be laproscopically implanted into a human abdominal cavity such that a side of the expandable barrier interfaces with organs in the abdominal cavity, thereby forming a barrier between the organs and a region of the abdominal cavity on an opposing side of the barrier relative to the organs,
wherein at least a portion of the barrier is configured to be controllably transformed from a first state to a second state, wherein when in the second state the barrier is in a more rigid state relative to the first state.

2. The device of claim 1, wherein at least a portion of the barrier is inflatable such that inflation of the inflatable portion transforms the barrier from the first state to the second state.

3. The device of claim 1, wherein the inflatable portion of the barrier is located at about a central region of the barrier.

4. The device of claim 1, wherein the barrier is configured to receive a fluid such that introduction of a fluid into the barrier when barrier is in the first state transforms the barrier from the first state to the second state.

5. The device of claim 4, further comprising:
at least one valve, wherein the valve is configured to control ingress of fluid to the barrier.

6. The device of claim 1, further comprising:
a valve system configured to transform the barrier from the first state to the second state in a incremental manner.

7. The device of claim 6, wherein:
the valve system is configured to permit a user to transform the barrier from the first state to a state in between the first state and the second state having a rigidity in between the first state and the second state and to maintain the barrier at the state in between the first state and the second state.

8. The device of claim 1, wherein:
the second state is an expanded state; and
the first state is a collapsed state.

9. The device of claim 8, wherein:
the barrier is configured such that, when in the second state, the barrier forms a bowl shape which retains organs therein.

10. The device of claim 4, wherein:
the barrier is configured such that respective shapes of the barrier when in the first state and the second state are dependent on an amount of fluid in the barrier.

11. The device of claim 1, wherein the barrier includes an elastomer body in which fiber reinforcement is embedded.

12. The device of claim 4, further comprising channels configured to receive the fluid such that introduction of the fluid into the channels when barrier is in the first state transforms the barrier from the first state to the second state, wherein;
the channels are formed in an elastomer in which fiber reinforcement is embedded.

13. A device for human organ packing in a laproscopic surgical procedure, comprising:
an expandable and collapsible barrier configured to be laproscopically positioned into a human such that a side of the barrier interfaces with organs, thereby forming a barrier between the organs and a region on an opposing side of the barrier relative to the organs, wherein a rigidity of the barrier is configured to be controllably adjusted.

14. The device of claim 13, wherein:
at least a portion of the barrier is inflatable such that inflation of the portion provides rigidity to the barrier.

15. The device of claim 13, wherein:
the at least a portion of the barrier that is inflatable is located at about a central region of the barrier.

16. The device of claim 13, wherein:
the barrier is configured to receive a fluid such that addition of the fluid into the barrier provides rigidity to the barrier.

17. The device of claim 16, further comprising:
at least one valve, wherein the valve is configured to control ingress of fluid to the barrier.

18. The device of claim 16, further comprising:
a valve system configured to increase rigidity of the barrier in an incremental manner.

19. The device of claim 18, wherein:
the valve system is configured to permit a user to adjust the rigidity of the barrier from a less rigid state to a more rigid state without further imparting rigidity to the barrier; and
the valve system is configured to permit a user to optionally subsequently adjust the rigidity of the barrier to have even more rigidity than that of the more rigid state.

20. The device of claim 13, wherein:
the expandable and collapsible barrier is configured to be laproscopically positioned into a human abdominal cavity such that a side of the barrier interfaces with organs in the abdominal cavity, thereby forming a barrier between the organs and a region of the abdominal cavity on an opposite side of the barrier relative to the organs.

21. The device of claim 13, wherein:
at least a portion of the barrier is inflatable such that inflation of the portion provides rigidity to the barrier; and
at least a portion of the barrier that is inflatable is formed from an elastomer body in which fiber reinforcement is embedded.

22. A laproscopic surgery procedure including human organ packing, comprising:
forming a laproscopic incision in the human;
inserting a tube through the incision such that a distal end of the tube is disposed in the abdominal cavity of the patient;
moving a flexible barrier in a collapsed state out of the distal end of the tube so that the flexible barrier is disposed in the abdominal cavity;
remotely flowing fluid to the barrier to transform the barrier from the flexible state to a more rigid state; and
positioning the barrier in the abdominal cavity such that the barrier provides a barrier between the organs and a portion of the abdominal cavity on an opposing side of the barrier relative to the organs.

23. The laproscopic surgery procedure of claim 22, further comprising:
applying a sufficient force to the barrier, when in the flexible state, to cause the barrier to move from the tube and into the abdominal cavity.

24. A device for human organ packing in a laproscopic surgical procedure, comprising:
an expandable barrier configured and dimensioned to, in an expanded state:
be located in a human abdominal cavity such that a side of the expandable barrier interfaces with the organs in the abdominal cavity, thereby forming a barrier between the organs and the portions of the abdominal cavity on an opposing side of the barrier relative to the organs;
wherein the barrier is configured to receive a fluid such that introduction of a fluid into the barrier when the expandable barrier is in a collapsed state causes at least a portion of the expandable barrier to expand.

25. The device of claim 24, wherein:
the barrier is configured to be moved through a cannula of a trocar and into an abdomen by a structure connected to the barrier.

26. The device of claim 25, wherein:
the structure is a rigid elongate structure.

27. The device of claim 24, further comprising:
a tube extending from the expandable barrier, the tube configured to communicate fluid to at least a portion of the expandable barrier to expand the barrier via the fluid introduction therein.

28. The device of claim 27, wherein:
the tube connects to the barrier at about a central position thereof

29. The device of claim 27, wherein:
the expandable barrier is configured to be inserted through a trocar and/or a device having a structure generally corresponding to a structure of a trocar medical barrier, and wherein the tube is configured to extend through the trocar and/or the device having a structure generally corresponding to a structure of a trocar medical barrier.

30. The device of claim 27, wherein:
the tube is of sufficient structural rigidity to withstand a vacuum that removes fluid from the barrier after the fluid has been received in the barrier to collapse the barrier from the expanded state.

31. The device of claim 24, wherein:
the barrier is configured to be moved from the abdomen and through a cannula of a trocar by a structure connected to the barrier having substantial length as compared to its thickness.

32. The device of claim 25, wherein:
the structure is a tube extending from the expandable barrier and configured to extend through a cannula of the trocar; and
the tube is configured to communicate fluid to the expandable barrier to expand the barrier via the fluid introduction therein.

33. The device of claim 31, wherein:
the structure is a rigid elongated structure.

34. An assembly, comprising:
the device of claim 29; and
the trocar and/or the device having a structure generally corresponding to a structure of a trocar medical barrier, wherein the trocar and/or the device having a structure generally corresponding to a structure of a trocar medical barrier includes:
an elongated extension having an inner lumen; and
a projection extending away from the elongated extension at a proximal end of the elongated extension.

35. The device of claim 24, further comprising:
at least one valve, wherein the valve is configured to control ingress of fluid to the barrier.

36. The device of claim 24, further comprising:
a valve system configured to expand the barrier in an incremental manner.

37. The device of claim 36, wherein:
the valve system is configured to permit a user to expand the barrier from a collapsed state to a partially expanded state and maintain the barrier in the partially expanded state without further expansion.

38. The device of claim 24, further comprising:
a support structure, wherein the support structure is inflatable.

39. The device of claim 38, wherein:
the support structure is located at about a central portion of the barrier.

40. The device of claim 24, wherein:
the barrier includes non-uniform surface features on the side of the barrier that contacts the organs.

41. The device of claim 40, wherein:
the non-uniform surface features correspond to protrusions having a circular cross-section.

42. The device of claim 24, wherein:
the barrier includes areas of relative roughness on the side of the barrier that contacts the organs.

43. The device of claim 24, wherein:
the barrier has an outer extrapolated circumference when in the expanded state; and
the barrier has openings located within the outer extrapolated circumference.

44. The device of claim 24, wherein:
the barrier is configured to be rolled and/or folded to fit into a cannula of a trocar.

45. The device of claim 24, wherein:
the barrier is configured to be unrolled and/or unfolded to expand once inside the abdominal cavity.

46. The device of claim 24, wherein:
the barrier is configured such that, when expanded, the barrier forms a bowl shape which retains organs therein.

47. The device of claim 24, wherein:
the barrier is configured such that a final shape of the barrier, as a result of expansion, is dependent on an amount of fluid in the barrier.

48. A laproscopic surgery procedure including human organ packing, comprising:
forming a laproscopic incision in the human;
inserting a tube through the incision such that a distal end of the tube is disposed in the abdominal cavity of the human;
moving a collapsed flexible barrier out of the distal end of the tube so that the collapsed barrier is disposed in the abdominal cavity;
remotely uncollapsing the barrier in the abdominal cavity by flowing fluid to the barrier; and
positioning the barrier in the abdominal cavity such that the barrier provides a barrier between the organs and a portion of the abdominal cavity on an opposing side of the barrier relative to the organs.

49. The laproscopic surgery procedure of claim 48, further comprising:
applying a sufficient force to the collapsed barrier to cause the barrier to move from the tube and into the abdominal cavity.

50. The laproscopic surgery procedure of claim 48, further comprising:
moving the collapsed barrier out of the end of the tube using a rigid structure attached to the collapsed barrier.

51. The laproscopic surgery procedure of claim 48, further comprising:
controlling the flow of fluid into the barrier to incrementally uncollapses the barrier.

52. The laproscopic surgery procedure of claim 48, wherein:
controlling the flow of fluid into the barrier is executed using at least one valve.

53. The laproscopic surgery procedure of claim 48, further comprising:
controlling an uncollapsing size of the barrier by controlling an amount of fluid communicated to the barrier.

54. The laproscopic surgery procedure of claim 48, further comprising:
manipulating the barrier with tools extending through the incision while the barrier is in the abdominal cavity.
